(19) United States
(12) Patent Application Publication
(10) Pub. No.: US 2012/0130045 A1
(43) Pub. Date: May 24, 2012

(54) BIOMOLECULE-POLYMER CONJUGATES AND METHODS OF MAKING SAME

(75) Inventors: Nicholas Lee Hammond, Hattiesburg, MS (US); Lisa Kay Kemp, Hattiesburg, MS (US); Tyler Weis Hodges, Hattiesburg, MS (US)

(73) Assignee: ABLITECH, INC., Hattiesburg, MS (US)

(21) Appl. No.: 13/375,302

(22) PCT Filed: Jun. 1, 2010
(86) PCT No.: PCT/US10/36957

§ 371 Date: Jan. 17, 2012

Related U.S. Application Data
(60) Provisional application No. 61/182,838, filed on Jun. 1, 2009.

(51) Int. Cl. C07H 21/04; C07K 14/805; B32B 1/02; C08B 37/08; C07H 19/20; C07H 21/02; C07K 14/435

(52) U.S. Cl. 530/324; 536/24.5; 530/385; 536/20; 536/26.26; 536/23.1; 428/34.1

(57) ABSTRACT

Disclosed herein are biomolecule-polymer conjugates of Formula 1, as well as methods of preparing same and kits for preparing same.

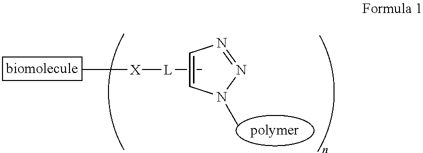

This application claims the benefit of U.S. Provisional Application No. 61/182,838, filed Jun. 1, 2009, which is herein incorporated by reference in its entirety.

### BACKGROUND

Recent advancements in the identification of biologically relevant targets has led to the discovery of pharmaceutically and/or therapeutically useful biomolecules, for examples small biomolecules (lipids, phospholipids, glycolipids, sterols, vitamin, hormones, neurotransmitters, carbohydrates, sugars, disaccharides, natural products), bimolecular monomers (amino acids, nucleotides, monosaccharides), and bimolecular polymers (peptides, oligopeptides, polypeptides, proteins, ribonucleic acids (RNA), deoxyribonucleic acids (DNA), oligosaccharides, polysaccharides, and lignin). For example, selective genetic modifying agents such as small interfering RNA (siRNA) and micro RNA (mRNA) have been proposed and developed. Though the identification of biologically relevant targets has grown considerably, significant issues have plagued the use of such biomolecules, particularly the ability to provide degradative stability, deliver such biomolecules to their site of action in vivo, and recover efficacy.

Among the particular problems is the instability of the biomolecules. Such biomolecules are generally susceptible to environmental degradation in vitro and enzymatic degradation in vivo. Furthermore, the inability to selectively deliver the biomolecules to their site of action has limited their therapeutic utility.

Attempts to solve these pervasive problems have been plagued with further problems. For example, attempts have been made to chemically modify the biomolecules to prevent degradation, to make use of viral technology to provide site-specific delivery, and to encapsulate the biomolecules within polymer structures. These attempted solutions, however, typically lower the efficacy of the biomolecules, cause significant toxic side effects, or fail to achieve the site-specific delivery required to achieve efficacious results. The solution of this vexing problem would thus address a longfelt need in the field of biomolecular pharmaceutics.

The problems discussed above are particularly relevant in the field of small interfering RNA (siRNA). Small interfering RNA refers to RNA oligonucleotides that modulate protein expression. Small interfering RNAs offer great potential in the treatment of numerous diseases, such as cancer, but have failed to reach their full potential due to an inability to reach the site of action in an active form. See R. James Christie et al Endocrinology, 2010, 151(2), 466-473.

RNA interference (RNAi) generally refers to a pathway in eukaryotic cells for sequence-specific targeting and cleavage of complementary messenger RNA. See S. M. Elbashir et al., Nature, 2001, 411, 494-498. This is accomplished through the delivery of complementary strands of DNA or RNA into cells, complexation of these strands with proteins or enzymes that allow for the degradation or inhibition of mRNA thereby inhibiting cellular mechanisms.

Currently, there have been no siRNAs successfully commercialized, at least in part because of the delivery problems described above. There are currently two basic technologies for siRNA delivery in clinical trials. The first involves local delivery of an siRNA to the site of action to treat maladies such as age-related macular degeneration (AMD). See K. A. Whitehead, K. A. et al., Nature Reviews Drug Discovery, 2009, 8, 129-138. This technique does not utilize a protective mechanism to prevent the degradation of the siRNA or provide a selective targeting mechanism to increase the specificity of the siRNA delivery, and as such does not address the above-described problems. Indeed, this delivery technology is limited to local administration of therapeutically high doses of the siRNA.

The second technology currently in clinical trials involves the use of viral vectors for siRNA transport. See T. R. Brummelkamp et al., Cancer Cell, 2002, 2(3), 243-247 While it has been found that viral vectors are effective for in vitro delivery, significant additional problems arise from this technology. Specifically, use of viral vectors in early clinical trials has led to multiple adverse side effects and even death. The severity and unpredictability of viral vectors for therapeutic use in the general population has yet to be determined.

A potential solution to the siRNA delivery problem involves the direct modification of the siRNA to prevent environmental and enzymatic degradation. The methods used to date, however, suffer from several drawbacks: although modification can increase the stability, a substantial reduction of efficacy has been observed.

It is thus an object of the disclosure to provide a method of stabilizing biomolecules, for example siRNAs, by modifying the biomolecules to provide biomolecule conjugates that solve the above-described problems. Further, it is an object of the disclosure to provide biomolecule conjugates that have enhanced stability yet retain their efficacy.

### SUMMARY

In one aspect of the disclosure, provided herein is biomolecule-polymer conjugate(s) of Formula 1:

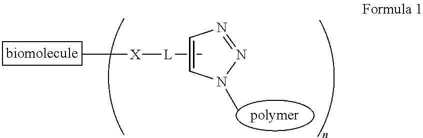

where the linker L is independently a 1-20 atom linear or branched linker; the polymer is independently a biocompatible polymer; X is independently an atom of attachment to the biomolecule that is O, NH, NR, or S, where R is part of the biomolecule; n is an integer from about 1 to about 30; and the X-L bond is degradable. The L-triazole bond can be to either carbon of the triazole ring, and is represented by the loose bond as illustrated in Formula 1. In certain embodiments, the biomolecule of Formula 1 is a nucleotide, nucleic acid, polynucleotide, amino acid, peptide, polypeptide, protein, or polysaccharide.

In another aspect of the disclosure, provided herein is a method of preparing a biomolecule-polymer conjugate(s) of Formula 1. The method comprises: (a) reacting the biomolecule with an alkyne-containing electrophilic reagent, and (b) reacting the alkyne-modified biomolecule with an azide-containing polymer or mixture of azide-containing polymers. The reaction is illustrated in Scheme 1 below:

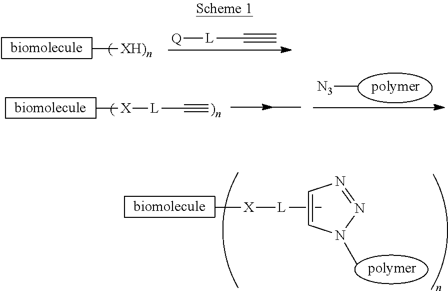

where the polymer, X, L, and n are as defined above, and Q is a leaving group.

In another aspect of the disclosure, provided herein is a kit suitable for preparing a biomolecule-polymer conjugate(s) of the disclosure, the kit comprising an alkyne-containing electrophilic reagent in a first container, an azide-containing biocompatible polymer in a second container, and instructions for their use.

The invention is based, in part, on the surprising and unexpected discovery of biomolecule-polymer conjugates that have increased stability compared to unmodified biomolecules, yet retain their efficacy against their intended target.

Those skilled in the art will realize that this invention is capable of embodiments that are different from those shown and details of the devices and methods can be changed in various manners without departing from the scope of this invention. Accordingly, the drawings and descriptions are to be regarded as including such equivalent embodiments as do not depart from the spirit and scope of this invention.

### BRIEF DESCRIPTION OF DRAWINGS

For a more complete understanding and appreciation of this invention, and its many advantages, reference will be made to the following detailed description taken in conjunction with the accompanying drawings.

FIG. 1A illustrates an embodiment of the first step of the method described herein, wherein an adenine amino group is reacted with propargyl chloroformate.

FIG. 1B illustrates an embodiment of the first step of the method described herein, illustrating modification of one or more amino and hydroxyl groups of various nucleobases.

FIG. 2 illustrates an embodiment of the second step of the method described herein, where an alkyne-containing biomolecule, the product of the first step of the method, is reacted with an azide-containing polymer, to form a biomolecule-polymer conjugate(s) of the disclosure.

FIG. 3A illustrates an embodiment of a biomolecule-polymer conjugate(s) of the disclosure where an siRNA is conjugated to multiple azide-containing polymers.

FIG. 3B illustrates an embodiment of a biomolecule-polymer conjugate(s) of the disclosure where an alkyne-modified biomolecule is conjugated to multiple azide-containing polymers.

FIG. 3C illustrates an embodiment of a biomolecule-polymer conjugate(s) of the disclosure where an siRNA is conjugated to multiple azide-containing polymers terminated with a functional group.

FIG. 4 illustrates an embodiment of a biomolecule-polymer conjugate(s) of the disclosure that is a biomolecule-polymer conjugate network.

FIG. 5 shows Thin Layer Chromatography (“TLC”) results under ultraviolet (“UV”) light showing deoxyribonuclease (“DNase”) I digestion after one hour of an oligonucleotide-MPEG conjugate prepared from methoxy-polyethylene glycol with an average molecular weight of 550 (“MPEG550”), the unmodified oligonucleotide, and a blend of the unmodified oligonucleotide and MPEG550.

FIG. 6 shows TLC results under UV light showing DNase I digestion after six hours of the conjugate and unmodified oligonucleotide of FIG. 5.

FIG. 7 shows TLC results under UV light showing DNase I digestion after 3 hours of the oligonucleotide-MPEG conjugate and an oligonucleotide-MPEG conjugate treated with NH₄OH to chemically remove the MPEG550.

FIG. 8 shows TLC results under UV light (left) and vanillin stained (right) showing DNase I digestion after 48 hours of functional K-ras sequence, functional K-ras sequence treated with an alkyne-containing reagent, and functional K-ras sequence conjugated with approximately one stoichiometric equivalent of MPEG6k, functional K-ras sequence conjugated with approximately six stoichiometric equivalents of MPEG6k, and functional K-ras sequence conjugated with a large stoichiometric excess of MPEG6k.

FIG. 9 shows TLC results under UV light (left) and vanillin stained (middle) showing DNase I digestion after one hour of polymerase chain reaction (“PCR”) primer (control), PCR primer (digest), a PCR primer-MPEG550 conjugate of the disclosure, and PCR primer-MPEG550 networked conjugate of the disclosure; and shows gel electrophoresis results (right) in a 1% agarose gel showing the PCR amplification products of PCR primer (unmodified 8F primer), PCR primer-MPEG550 conjugate, and PCR primer-MPEG550 conjugate treated with NH₄OH for either 15 minutes and 18 hours to chemically remove the MPEG550.

FIG. 10 shows TLC results under UV light (left) and vanillin stained (right) showing S1 Nuclease digestion after 30 minutes of Salmon sperm (“SS”) DNA (control) and a SS DNA-MPEG550 conjugate of the disclosure.

FIG. 11 shows TLC results under UV light showing Fetal Calf Serum (“FCS”) digestion after 36 hours with samples including a functional p53 siRNA (control), a functional p53 siRNA-MPEG550 conjugate of the disclosure (control), a functional p53 siRNA (digest), and a functional p53 siRNA-MPEG550 conjugate of the disclosure (digest).

### DETAILED DESCRIPTION

Generally, the nomenclature used herein and the laboratory procedures in organic chemistry, medicinal chemistry, and pharmacology described herein are those well known and commonly employed in the art. Unless defined otherwise, all technical and scientific terms used herein generally have the same meaning as commonly understood by one of ordinary skill in the art to which this disclosure belongs. In the event that there is a plurality of definitions for a term used herein, those in this section prevail unless stated otherwise.

The term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 20%, 15%, 10%, 9%, 8%, 7%, 6%, 5%, 4%, 3%, 2%, 1%, 0.5%, or 0.05% of a given value or range.

The term “optionally substituted” is intended to mean that a group, such as an alkyl, alkenyl, alkynyl, cycloalkyl, aryl, heteroaryl, heterocyclyl, or alkoxy group, may be substituted with one or more substituents independently selected from, e.g., (a) alkyl, alkenyl, alkynyl, cycloalkyl, aryl, heteroaryl, and heterocyclyl, each optionally substituted with one or more, in one embodiment, one, two, three, or four, substituents Q; and (b) halo, cyano (—CN), nitro (—NO₂), —C(O)R^{a}, —C(O)OR^{a}, —C(O)NR^{b}R^{c}, —C(NR^{a})NR^{b}R^{c}, —OR^{a}, —OC(O)R^{a}, —OC(O)OR^{a}, —OC(O)NR^{b}R^{c}, —OC(═NR^{a})NR^{b}R^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(═NR^{d})NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —SR^{a}, —S(O)R^{a}, —S(O)₂R^{a}, —S(O)NR^{b}R^{c}, and —S(O)₂NR^{b}R^{c}, wherein each R^{a}, R^{b}, R^{c}, and R^{d} is independently (i) hydrogen; (ii) C₁₋₆ alkyl, C₂₋₆ alkenyl, C₂₋₆ alkynyl, C₃₋₇ cycloalkyl, C₆₋₁₄ aryl, heteroaryl, or heterocyclyl, each optionally substituted with one or more, in one embodiment, one, two, three, or four, substituents Q; or (iii) R^{b} and R^{c} together with the N atom to which they are attached form heterocyclyl, optionally substituted with one or more, in one embodiment, one, two, three, or four, substituents Q. As used herein, all groups that can be substituted are “optionally substituted,” unless otherwise specified.

“Biocompatible” refers to being compatible with a living tissue, by virtue of, e.g., low or no toxicity, or no immunological rejection. In certain embodiments, a polymer is biocompatible if it has good safety ratio or therapeutic index or protective index. In certain embodiments, a polymer is biocompatible if it has been approved for use in humans by any regulatory agency, such as the FDA or EMEA.

“Biomolecule” means any organic molecule. In an embodiment, a biomolecule is an organic molecule produced by a living organism or an analog or derivative thereof. A biomolecule can include a biomolegical molecule. Biomolecules include, but are not limited to, lipids, phospholipids, glycolipids, sterols, vitamins, hormones, neurotransmitters, carbohydrates, sugars, disaccharides, amino acids, nucleotides, nucleosides, polynucleotides, saccharides (mono-, poly-, or oligo-saccharides), peptides, polypeptides, proteins, nucleic acids (e.g., ribonucleic acids (RNA), deoxyribonucleic acids (DNA), as well as nucleic acid analogs thereof and polymeric forms thereof), lignin, and mixed groups thereof. In certain embodiments, a biomolecule includes a peptide, polypeptide, protein, lipid, sugar, polysaccharide, nucleic acid, nucleotides, or polynucleotides, as well as well as derivatives of the above comprising amino acid or nucleotide analogs or other non-nucleotide groups. Biomolecule encompasses those in which the conventional polynucleotide backbone has been replaced with a non-naturally occurring or synthetic backbone, and those in which one or more of the conventional bases has been replaced with a synthetic base capable of participating in Watson-Crick type hydrogen bonding interactions. Polynucleotides include single or multiple strand configurations, where one or more of the strands may or may not be completely aligned with one another. Nucleic acid analogs include, for example and without limitation, phosphorothioates, phosphorodithioates, phosphorotriesters, phosphoramidates, boranophosphates, methylphosphonates, chiral-methyl phosphonates, 2-O-methyl ribonucleotides, peptide-nucleic acids (PNAs), locked-nucleic acids (LNAs), and the like. In some embodiments, a biomolecule is cDNA, siRNA, microRNA, short hairpin RNA, piwi-interacting RNAs (piRNAs), mitrons, antisense molecules, or another oligonucleotide.

A “nucleotide” refers to a subunit of a nucleic acid and includes a phosphate group, a 5 carbon sugar and nitrogen containing base, as well as analogs of such subunits. An “oligonucleotide” generally refers to a nucleotide multimer of about 10 to 100 nucleotides in length, while a “polynucleotide” includes a nucleotide multimer having any number of nucleotides.

“Degradable” means covalent bonds capable of being broken via hydrolysis (reaction with water) under basic or acid conditions, via metabolic pathways, enzymatic degradation (by environmental and/or physiological enzymes), or other biological processes (such as those under physiological conditions in a vertebrate, such as a mammal). A degradable bond includes, but is not limited to, carboxylate esters, phosphate esters, carbamates, anhydrides, acetals, ketals, imines, orthoesters, thioesters, or carbonates.

“Targeting group” means those moieties that have been shown to influence the accumulation of a biomolecule in specific cells. Targeting groups can be comprised of a variety of proteins, peptides, small molecules, or the like. Non-limiting examples include vitamin D and folate (e.g., for cancer cells).

### Biomolecule-Polymer Conjugates

The biomolecule-polymer conjugate(s) of the disclosure are illustrated in Formula 1:

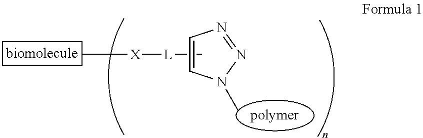

where the linker L is independently a 1-20 atom linear or branched linker; the polymer is independently a biocompatible polymer; X is independently an atom of attachment to the biomolecule that is O, NH, NR, or S, where R is part of the biomolecule; n is an integer; and the X-L bond is degradable. The loose bond between “L” and the triazole in Formula 1 indicates that the linker “L” can be bound to either carbon of the triazole ring.

In certain embodiments, the biomolecule of Formula 1 is a nucleotide, nucleic acid, polynucleotide, amino acid, peptide, polypeptide, protein, or polysaccharide. In certain embodiments, the biomolecule of Formula 1 is a DNA, RNA, peptide, polypeptide, protein, polysaccharide, nucleic acid, nucleotide, amino acid, or polynucleotide. In certain embodiments, the biomolecule is an RNA or DNA oligonucleotide, for example an antisense RNA or DNA oligonucleotide. In a particular embodiment, the biomolecule is an siRNA. In certain embodiments, the biomolecule is a mixed group of any of the above.

In certain embodiments, the biomolecule is an RNA or DNA oligonucleotide. In a specific embodiment, the biomolecule is siRNA, mRNA, mitron, microRNA, or antisense. In certain subembodiments, the oligonucleotide comprises from about 2 to about 30 bases, from about 5 to about 25 base pairs, or from about 10 to about 25, or from about 15 to about 25 bases.

In certain embodiments, the polymer of Formula 1 is a biocompatible polymer. In certain embodiments, the polymer imparts a stabilizing effect on the biomolecule. When n is greater than 1, the various polymers of Formula 1 can be the same or different. In various embodiments, the polymer is independently anionically charged, cationically charged, or uncharged; hydrophobic, hydrophilic, or amphiphilic; or combinations thereof. In various embodiments, the polymer is a homopolymer, a block copolymer, or a random copolymer. In certain embodiments, the polymer is polydisperse or monodisperse. In various embodiments, the polydispersity index of the polymer is from 1 to about 30, from 1 to about 10, from 1 to about 5, or from 1 to about 3. In certain embodiments the polymer is linear. In certain embodiments the polymer is branched.

In certain embodiments, In certain embodiments, the polymer is a polyethylene glycol (PEG), a polyether, a poly(lactide), a poly(glycolide), a poly(lactide-co-glycolide), a poly(lactic acid), a poly(glycolic acid), a poly(lactic acid-co-glycolic acid), a polyanhydride, a polyorthoester, a polycarbonate, a polyetherester, a polycaprolactone, a polyesteramide, a polyester, a polyacrylate, a polymer of ethylene-vinyl acetate or another acyl substituted cellulose acetate, a polyurethane, a polyamide, a polystyrene, a silicone based polymer, a polyolefin, a polyvinyl chloride, a polyvinyl fluoride, a fluoropolymer, a polypropylene, a polyethylene, a cellulosic, a starch, a naturally occurring polymer, a poly(vinyl imidazole), a polyacetal, a polysulfone, a chlorosulphonate polyolefin, or a blend or copolymer thereof. In a particular embodiment, the polymer is PEG.

In various embodiments, the polymer of Formula 1 has an average molecular weight of from about 200 to about 50,000, from about 200 to about 40,000, from about 200 to 30,000, from about 200 to about 20,000, from about 200 to about 10,000, from about 200 to about 5,000, from about 200 to about 4,000, from about 200 to about 3,000, from about 200 to about 2,000, from about 200 to about 1,000, or from about 200 to about 500. In various embodiments, the polymer has an average molecular weight of from about 10,000 to about 50,000, from about 10,000 to about 40,000, from about 10,000 to about 30,000, or from about 10,000 to about 20,000. In a particular embodiment, the polymer has an average molecular weight of from about 500 to about 5,000.

In certain embodiments, the polymer is independently terminated with a non-functional group, such as methyl or methoxy, or a functional group, such as a targeting group. In a particular embodiment, the targeting group is a folate. In certain embodiments, the polymer is terminated with another biomolecule. In such an embodiment, the biomolecule-polymer conjugate is a networked biomolecule-polymer conjugate, each conjugate comprising more than one biomolecule.

The linker “L” can of varying lengths and composition. In certain embodiments, the linker is from about 1 to about 20 atoms in length, from about 1 to about 15 atoms in length, from about 1 to about 10 atoms in length, or from about 1 to about 5 atoms in length. In certain embodiments, the linker is 1, 2, 3, 4, 5, or 6 atoms in length. In a particular embodiment, the linker is 3 atoms in length. In various embodiments, the atoms comprising the linker backbone are independently carbon, oxygen, nitrogen, or sulfur. In a particular embodiment, the linker L is: —C(O)O(CH₂)_{q}—, where q is an integer from 0 to about 20, from about 0 to about 10, from about 1 to about 10, from about 2 to about 10, from about 2 to about 8, from about 2 to about 5, or from about 2 to about 4. In various sub-embodiments, q is 0, 1, 2, 3, 4, 5, 6, 7, 8, 9, or 10. In a particular sub-embodiment, q is 2. In various sub-embodiments, each methylene group may be optionally substituted, or may itself be a different atom, such as NH, O, or S.

In certain embodiments, the L-X bond is degradable. In certain sub-embodiments, the degradable L-X bond is a carbonate bond, a carboxylate ester bond, a phosphate ester bond, an anhydride bond, an acetal bond, a ketal bond, an imine bond, an orthoester bond, a thioester bond a carbamate bond, a urea bond, an amide bond. In a particular sub-embodiment, the L-X bond in a carbonate or carbamate bond.

In certain embodiments, n is from about 1 to about 100, from about 1 to about 75, from about 1 to about 50, from about 1 to about 30 or from about 1 to about 20. In certain embodiments, n is from about 1 to about In various embodiments, n is from about 11 to about 30, from about 13 to about 27, from about 15 to about 25, or from about 17 to about 22. In various other embodiments, n is 1, 2, 3, 4, 5, 6, 7, 8, 9, 10, 11, 12, 13, 14, 15, 16, 17, 18, 19, 20, 21, 22, 23, 24, or 25. In a particular embodiment, n is from about 11 to about 14.

In certain embodiments, the biomolecule-polymer conjugate of the disclosure is degradable. This is advantageous in that the biomolecule-polymer conjugate may be initially stable for a period of time when introduced into a living system. This allows time for biomolecule-polymer conjugate to traverse harsh environments, such as the intestinal tract, circulatory system and liver, where the biomolecule alone could be trapped or degraded. The degradable nature of the biomolecule-polymer conjugate allows for the release of the biomolecule to the respective site of action in a living system full intact. The delay of degradation of the biomolecule-polymer conjugate allows for distribution to a variety of tissues and organs that would be less accessible by the biomolecule alone. In addition, the biomolecule-polymer conjugate may also allow for slow release of the biomolecule dependent on the rate of degradation. In certain embodiments, the biomolecule-polymer conjugate degrades in vivo to release the biomolecule with a half-life of less than about 2 weeks, less then about 1 week, less than about 2 days, less than about 1 day, less than about 12 hours, less than about 6 hours, or less than about 3 hours.

In certain embodiments, the biomolecule-polymer conjugate of the disclosure has enhanced stability compared to the corresponding unmodified biomolecule, for example in vivo stability as evidenced by, for example, circulation half-life.

In various embodiments, the biomolecule is released from the protecting polymer layer via degradation of a bond, e.g., the L-X bond, through which the biomolecule is conjugated to the polymer. In various embodiment, the degradation occurs via hydrolysis (reaction with water) under basic or acid conditions, metabolism, enzymatic degradation (by environmental and/or physiological enzymes), and other biological processes (such as those under physiological conditions in a vertebrate, such as a mammal). In embodiments where the degradation of the biomolecule-polymer conjugate generated acid functional groups (e.g., when the degradation occurs at an ester or carbonate bond), the degradation process provides an auto-catalytic effect.

In various embodiments, release of the biomolecule may involve the degradation of a biodegradable linker, or digestion of the polymer into smaller, non-polymeric subunits. Two different areas of biodegradation may occur: the cleavage of bonds in the polymer backbone which generally results in monomers and oligomers of the polymer; or the cleavage of a bond connecting the polymer to the biomolecule. In certain embodiments, the release of the biomolecule (e.g., the degradation of a bond linking the biomolecule to the polymer) occurs faster than the degradation of the biomolecule itself. The degradation rate can be measured both in vitro by known methods, for example by UV-Vis spectroscopy, or in vivo, by sampling blood serum over time and determining the concentration of the metabolits by known methods, for example HPLC.

The degradation rates of a bond linking the biomolecule to the polymer (such as the L-X bond) and of the polymer itself may vary.

In certain embodiments, the biomolecule-conjugate of the disclosure is useful for the treatment or prevention or amelioration of a disease, for the modulation of protein/mRNA expression, or as a diagnostic tool.

In another aspect of the disclosure, provided herein is a composition comprising a biomolecule-polymer conjugate(s) as described herein and a carrier.

### Methods of Preparing the Biomolecule-Polymer Conjugate

In another aspect of the disclosure, a method of preparing the biomolecule-polymer conjugates of Formula 1. The method comprises (a) reacting the biomolecule with an alkyne-containing electrophilic reagent, and (b) reacting the alkyne-modified biomolecule with an azide-containing polymer or mixture of azide-containing polymers. The reaction is illustrated in Scheme 1 below:

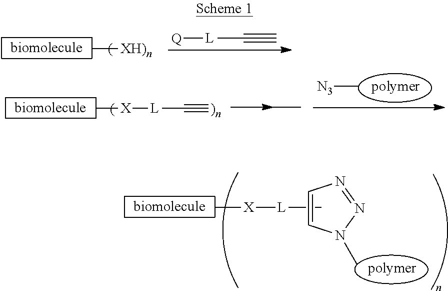

where the biomolecule, the polymer, X, L, and n are as defined above, and Q is a leaving group.

In certain embodiments, steps (a) and (b) are conducted as a “one-pot” synthesis, without isolation and/or purification of the intermediate alkyne-modified biomolecule. In other embodiments, steps (a) and (b) are conducted with isolation and/or purification of the intermediate alkyne-modified biomolecule.

In step (a) of the method, the biomolecule is reacted with an alkyne-containing electrophilic reagent to yield an L-X bond. In certain embodiments, the alkyne-containing electrophilic reagent is a carboxylic acid, an acid halide, a carboxylic acid anhydride, a carboxylic acid salt, a carboxylic acid ester, an isocyanate, a carbonate, a carbamate, or a chloroformate. In a certain embodiment, the alkyne-containing electrophilic reagent is

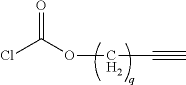

wherein q is an integer from 0 to about 20, from about 0 to about 10, from about 1 to about 10, from about 2 to about 10, from about 2 to about 8, from about 2 to about 5, or from about 2 to about 4. In various sub-embodiments, q is 0, 1, 2, 3, 4, 5, 6, 7, 8, 9, or 10. In a particular sub-embodiment, q is 2. In various sub-embodiments, each methylene group may be optionally substituted, or may itself be a different atom, such as NH, O, or S. In a particular embodiment, the alkyne-containing electrophilic reagent is a chloroformate, such as propargyl chloroformate.

In various embodiments, step (a) of the method proceeds via one or more of the following reactions (where R is the biomolecule, and X is either OH or NH₂):

Alcohol+propargyl Chloride, condensation reaction yields an carbonate bond

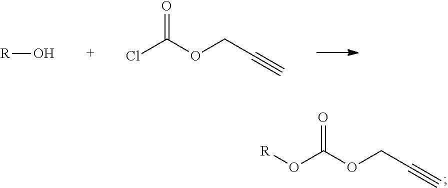

Alcohol+carboxylic acid, condensation reaction yields an ester bond

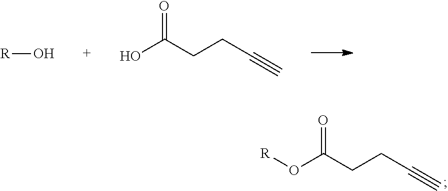

Alcohol+acid halide, condensation reaction yields an ester bond

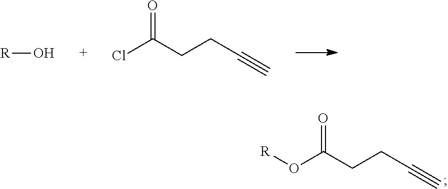

Alcohol+acid anhydride, condensation reaction yields an ester bond

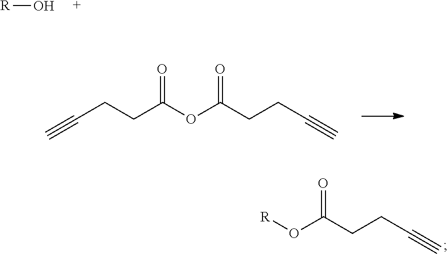

Alcohol+acid salts, condensation reaction yields an ester bond

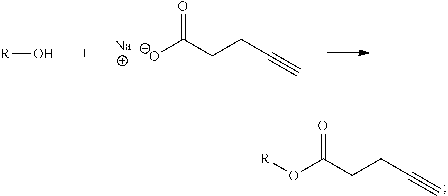

Alcohol+isocyanate, addition reaction yields a urethane bond

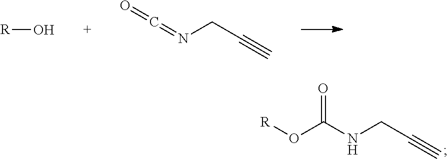

Alcohol+ester, transesterification reaction yields an ester bond

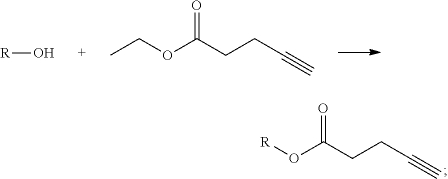

Amine+isocyanate, addition reaction yields a urea bond

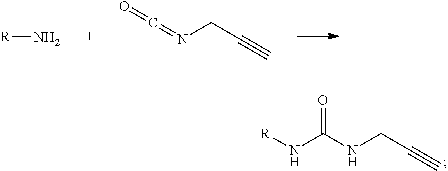

Amine+carboxylic acid, neutralization and dehydration reaction yields an amide bond

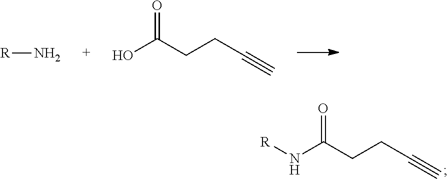

Amine+acid anhydride, substitution reaction yields an amide bond

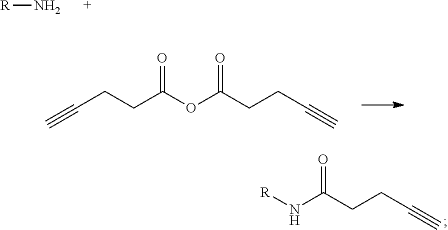

Amine+acid halide, substitution reaction yields an amide bond

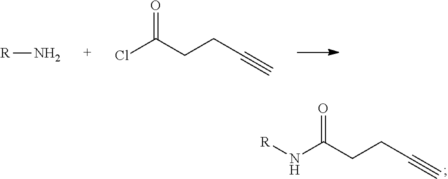

Amine+acid salts, reaction yields an amide bond

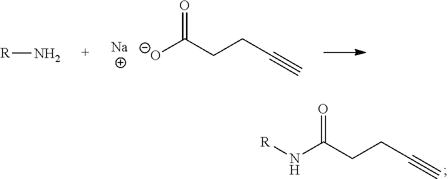

Amine+ester, reaction yields an amide bond

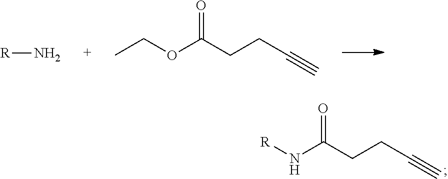

Amine+chloroformate, reaction yields a carbamate bond

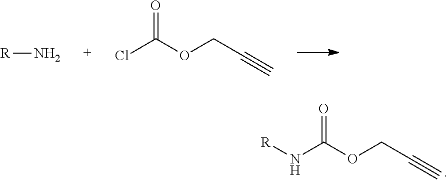

While the above exemplary reactions are illustrated where the alkyne 3 atoms away from the biomolecule atom (either the nitrogen or the oxygen), the disclosure encompasses other embodiments where the alkyne is anywhere from 1 to about 20 atoms away from the biomolecule atom. In other embodiments, the alkyne is from about 2 to about 10, or from about 2 to about 5 atoms away from the biomolecule atoms.

Step (a) of the can be conducted in a variety of solvents. In various embodiments, the first step of the method is conducted in water, tetraethylene glycol dimethylether, dimethylsulfoxide, dimethylformamide, chloroform, dichloromethane, pyridine, acetone, ether, or a mixture thereof. In a particular embodiment, the first step of the method is conducted in a mixture of water and one or more of tetraethylene glycol dimethylether, dimethylsulfoxide, dimethylformamide, chloroform, dichloromethane, pyridine, acetone, or ether. In certain embodiments, the reaction is conducted in the absence of water. In other embodiments, the reaction is conducted in water.

In certain embodiments, step (a) of the method is conducted in the presence of a base. In various embodiments, the base is a tertiary alkyl amine, an aromatic amine, a carbonate, or a hydroxide. In particular embodiments, the base is diisopropylethylamine, triethylamine, pyridine, sodium carbonate, potassium carbonate, sodium bicarbonate, potassium bicarbonate, sodium hydroxide, or potassium hydroxide.

Step (a) of the method can be conducted at a variety of temperatures and times, provided that the biomolecule is not degraded. In certain embodiments, the reaction is conducted at a temperature from about −30° C. to about 25° C., from about 0° C. to about 25° C., or from about 5° C. to about 20° C. In certain embodiments, the reaction is conducted for from about 5 minutes to about 8 hours, from about 5 minutes to about 1 hour, from about 20 minutes to about 40 minutes.

In certain embodiments, the biomolecule is treated with from about 0.001 to about 1000 molar equivalents of alkyne-containing electrophilic reagent based on the number of modifiable positions on the biomolecule. In various embodiments, the biomolecule is treated with from about 0.001 to about 1, from about 0.01 to about 1, from about 0.1 to about 1, or from about 0.5 to about 1 molar equivalent of alkyne-containing electrophilic reagent based on the number of modifiable positions on the biomolecule. In other embodiments, the biomolecule is treated with from about 1 to about 1000, from about 1 to about 500, from about 1 to about 100, from about 1 to about 10, or from about 1 to about 5 molar equivalents of alkyne-containing electrophilic reagent based on the number of modifiable positions on the biomolecule.

Optionally, the biomolecule can be treated prior to step (a) of the method. In certain embodiment, the pre-treatment is a desalting, denaturing, or splitting double stranded molecules into single strands.

In step (b) of the method, the alkyne-modified biomolecule is reacted with one or a mixture of azide-containing polymers. The azide-containing polymer can be any biocompatible polymer with an azide group. In certain embodiments, the azide-containing polymer imparts a stabilizing effect on the biomolecule. When n is greater than 1, the various polymers of Formula 1 can be the same or different. In various embodiments, the azide-containing polymer is independently anionically charged, cationically charged, or uncharged; hydrophobic, hydrophilic, or amphiphilic; or combinations thereof. In various embodiments, the azide-containing polymer is a homopolymer, a block copolymer, or a random copolymer. In certain embodiments, the azide-containing polymer is polydisperse or monodisperse. In various embodiments, the polydispersity index of the azide-containing polymer is from 1 to about 30, from 1 to about 10, from 1 to about 5, or from 1 to about 3. In certain embodiments the azide-containing polymer is linear. In certain embodiments the azide-containing polymer is branched.

In certain embodiments, the azide-containing polymer is a polyethylene glycol (PEG), a polyether, a poly(lactide), a poly(glycolide), a poly(lactide-co-glycolide), a poly(lactic acid), a poly(glycolic acid), a poly(lactic acid-co-glycolic acid), a polyanhydride, a polyorthoester, a polycarbonate, a polyetherester, a polycaprolactone, a polyesteramide, a polyester, a polyacrylate, a polymer of ethylene-vinyl acetate or another acyl substituted cellulose acetate, a polyurethane, a polyamide, a polystyrene, a silicone based polymer, a polyolefin, a polyvinyl chloride, a polyvinyl fluoride, a fluoropolymer, a polypropylene, a polyethylene, a cellulosic, a starch, a naturally occurring polymer, a poly(vinyl imidazole), a polyacetal, a polysulfone, a chlorosulphonate polyolefin, or a blend or copolymer thereof. In particular embodiments, the azide-containing polymer is PEG-azide.

In various embodiments, the azide-containing polymer has an average molecular weight of from about 200 to about 50,000, from about 200 to about 40,000, from about 200 to 30,000, from about 200 to about 20,000, from about 200 to about 10,000, from about 200 to about 5,000, from about 200 to about 4,000, from about 200 to about 3,000, from about 200 to about 2,000, from about 200 to about 1,000, or from about 200 to about 500. In various embodiments, the azide-containing polymer has an average molecular weight of from about 10,000 to about 50,000, from about 10,000 to about 40,000, from about 10,000 to about 30,000, or from about 10,000 to about 20,000. In a particular embodiment, the azide-containing polymer has an average molecular weight of from about 500 to about 5,000.

In certain embodiments, the azide-containing polymer is independently terminated with a non-functional group, such as a methyl or methoxy, or a functional group, such as a targeting group. In a particular embodiment, the targeting group is a folate. In certain embodiments, the azide-containing polymer is a mixture of non-functional terminated and functional terminated polymers. In certain embodiments, the mixture is a mixture of methoxy terminated and folate-terminated polymers, for example a mixture of methoxy-terminated PEG and folate-terminated PEG. In certain embodiments, the polymer is terminated with another biomolecule. In such an embodiment, the biomolecule-polymer conjugate is a networked biomolecule-polymer conjugate, each conjugate comprising more than one biomolecule.

Step (b) of the method can be conducted in a variety of solvents. In various embodiments, step (b) of the method is conducted in methanol, ethanol, propanol, isopropanol, tetraethylene glycol dimethylether, dimethylsulfoxide, dimethylformamide, acetone, ether, water, or a mixture thereof. In a particular embodiment, step (b) of the method is conducted in a mixture of water and one or more of methanol, ethanol, propanol, isopropanol, tetraethylene glycol dimethylether, dimethylsulfoxide, dimethylformamide, acetone, or ether. In a particular embodiment, step (b) of the method is conducted in water.

Step (b) of the method can be conducted at a variety of temperatures and times, provided that the biomolecule is not degraded. In certain embodiments, the reaction is conducted at a temperature from about −30° C. to about 70° C., from about 0° C. to about 65° C., or from about 25° C. to about 65° C. In certain embodiments, the reaction is conducted for from about 1 minute to about 8 hours, from about 5 minutes to about 3 hour, or from about 20 minutes to about 60 minutes.

In certain embodiments, step (b) of the method is conducted in the presence of a catalyst, for example in the presence of a copper catalyst. In a particular sub-embodiment of this embodiment, the copper catalyst is copper bromide or copper iodide. In certain embodiments, step (b) of the method is conducted in presence of a mixture of copper(II), e.g., copper(II) sulfate, and a reducing agent, e.g., sodium ascorbate.

In certain embodiments, step (b) of the method is conducted in the absence of a catalyst, for example in the absence of a metal catalyst such as copper. In these embodiments, the absence of a catalyst such as a copper catalyst may be particularly advantageous as the produced biomolecule-polymer conjugate is substantially free of copper. In various embodiment, the copper-free method produced a biomolecule-polymer conjugate that contains less than about 100 ppm copper, less than about 10 ppm copper, or less than about 1 ppm copper.

In certain embodiments, the method further comprises (c) purifying the biomolecule-polymer conjugate. In various embodiments, the conjugate is purified by size exclusion chromatography, reverse phase chromatography, thin layer chromatography, ion exchange chromatography, column chromatography, precipitation, or liquid-liquid extraction. In a particular embodiment, the conjugate is purified size exclusion chromatography.

In embodiments where the biomolecule is an RNA, modifiable nucleotides (i.e., adenine, guanine, cytosine, and uracil) are denoted in Formula 2. The reactive groups on the RNA include, but are not limited to, primary amines (i.e., where X in Formula 1 is NH), secondary amines (i.e., where X in Formula 1 is NR), and hydroxyl groups (i.e., where X in Formula 1 is O). In certain embodiments, only the primary amines and hydroxyl groups are modifiable. Secondary amines on natural RNAs are generally less reactive than primary amines, and thus may not always be modified in accordance with the method of the disclosure.

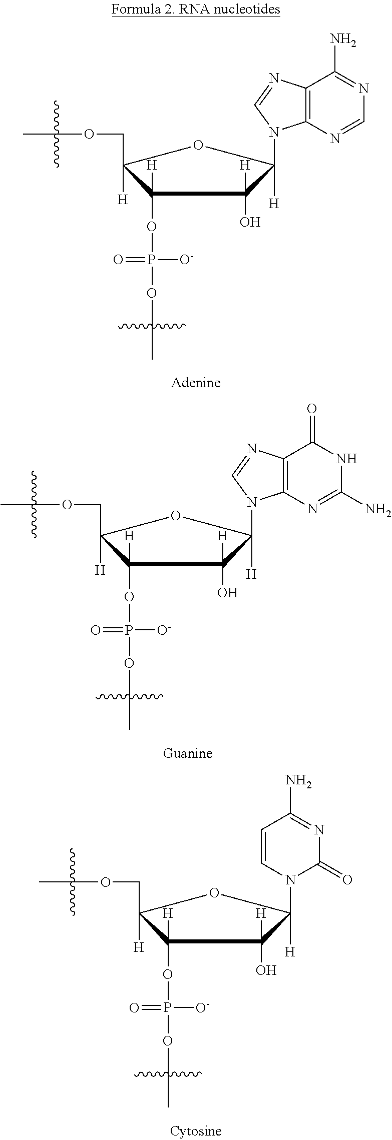

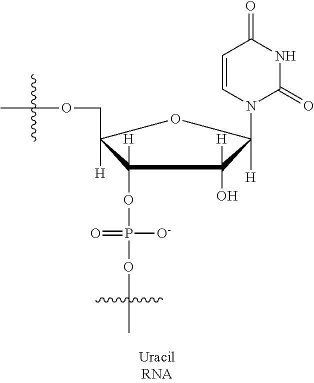

Specifically, the reactive moieties on the RNA nucleotides can be reacted with propargyl chloroformate. This reaction may be undertaken in a variety of different solvents or solvent mixtures. This reaction may also be undertaken in the presence or absence of bases, acids, acid scavengers, water scavenger, or drying reagents.

In embodiments where the biomolecule is an RNA and the alkyne-containing electrophilic reagent is propargyl chloroformate or the like, Formula 3 illustrate the modification of all reactive groups in each of the nucleotides. In other embodiments, the nucleotides are incompletely modified. It will be understood that each of the illustrated nucleotides is part of an oligonucleotide or polynucleotide chain, and thus the number of alkyne groups on a given RNA oligonucleotide or polynucleotide can vary. In various embodiments, the RNA comprises from about 1 to about 50, from about 5 to about 40, from about 10 to about 35, from about 10 to about 20, from about 20 to about 35, from about 10 to about 15, from about 15 to about 20, from about 20 to about 25, from about 25 to about 30, and from about 30 to about 35 alkyne groups after step (a) of the method.

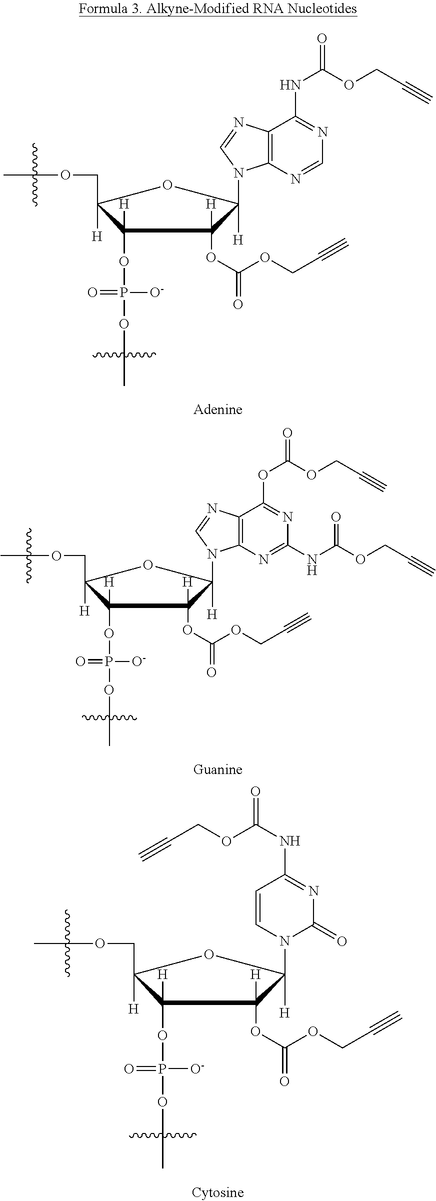

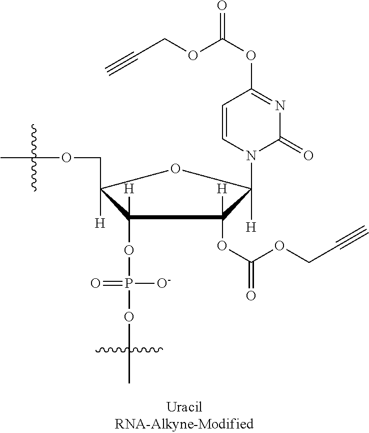

In embodiments where the biomolecule is an RNA and the alkyne-containing electrophilic reagent is propargyl chloroformate or the like, Formula 4 illustrates the product of the cycloaddition reaction between an azide-containing polymer (e.g., PEG azide terminated with a methoxy group or a targeting group) and the alkyne appended to an adenine. The alkyne group reacts with an azide end group of a polymer chain to form a triazole linkage. As seen in Formula 4, the resulting biomolecule-polymer conjugate exhibits a regioisomerism, that is there are two regioisomers formed at the triazole. This regioisomerism is illustrated by the loose bond in Formula 1.

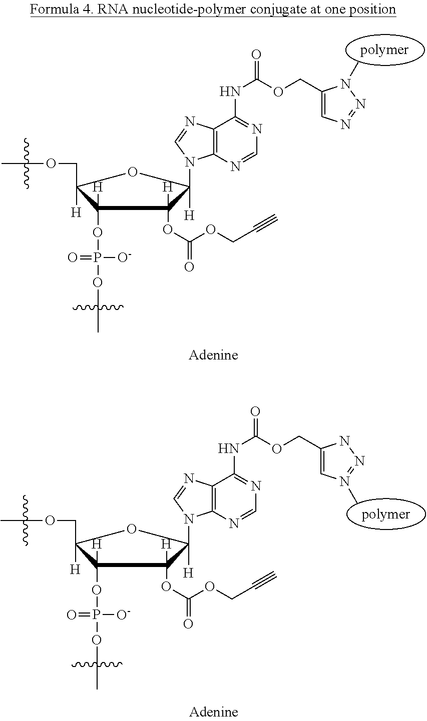

In embodiments where the biomolecule is an RNA and the alkyne-containing electrophilic reagent is propargyl chloroformate or the like, Formula 5 illustrates the product of the cycloaddition reaction between an azide-containing polymer (e.g., PEG azide terminated with a methoxy group or a targeting group) and two alkyne groups appended to an adenine. Each of the alkyne groups reacts with an azide end group of a polymer chain to form more than one triazole linkage As seen in Formula 5, the resulting biomolecule-polymer conjugate exhibits a regioisomerism, that is there are two regioisomers formed at each triazole. Thus, in an embodiment where there are two alkyne groups on one nucleobase, four regioisomers may be formed.

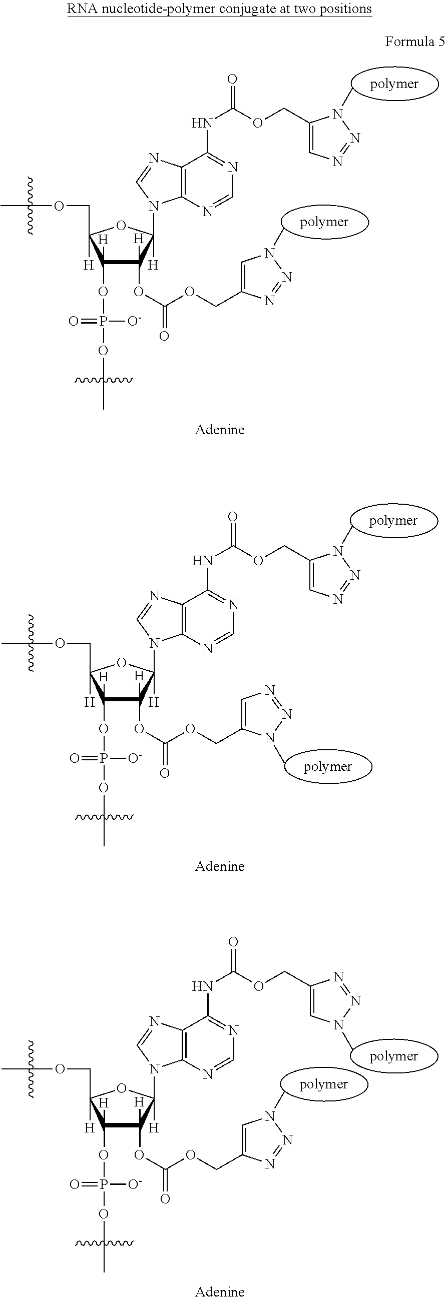

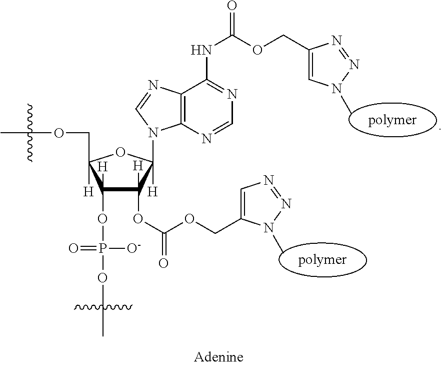

In embodiments where the biomolecule is an RNA and the alkyne-containing electrophilic reagent is propargyl chloroformate or the like, Formula 6 illustrates the product of the cycloaddition reaction between an azide-containing polymer (e.g., PEG azide terminated with a methoxy group or a targeting group) and one or two alkyne groups appended to guanine, cytosine, and uracil. As is also noted above, the modification of these nucleotides can embody single or multiple linkers to one or more polymer chains by forming one or more triazole rings as is illustrated in Formula 6.

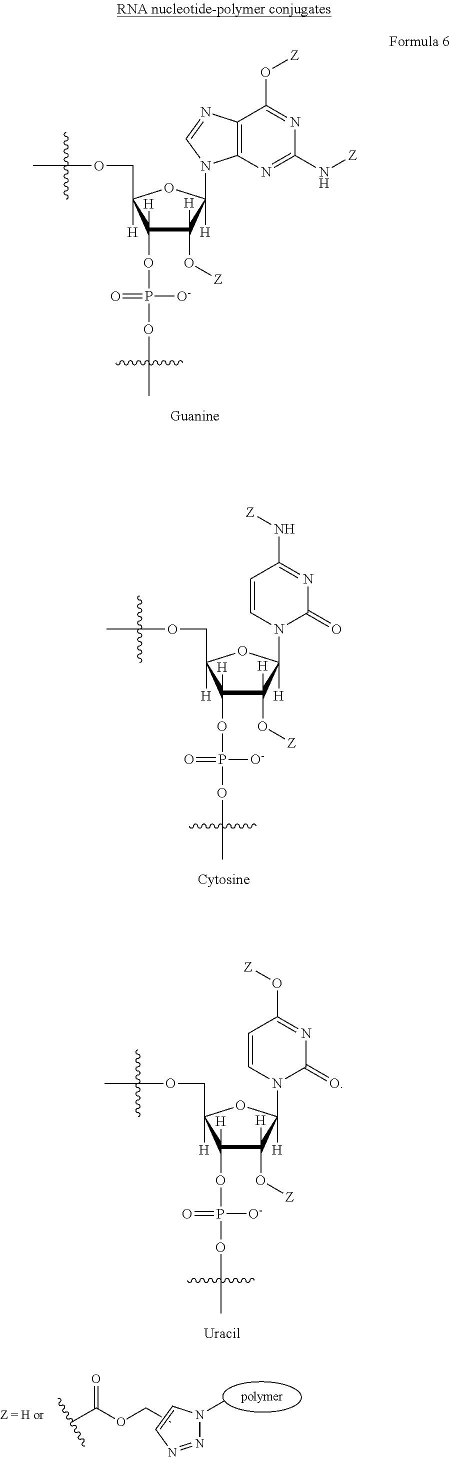

In embodiments where the biomolecule is an RNA and the alkyne-containing electrophilic reagent is propargyl chloroformate, the modification of the RNA may occur at the sugar hydroxyl only, as illustrated in Formula 7. Without intending to be limited by mechanism, it is believed that this mode of modification occurs for double-stranded oligonucleotides, where the base pairing precludes modification of the base itself.

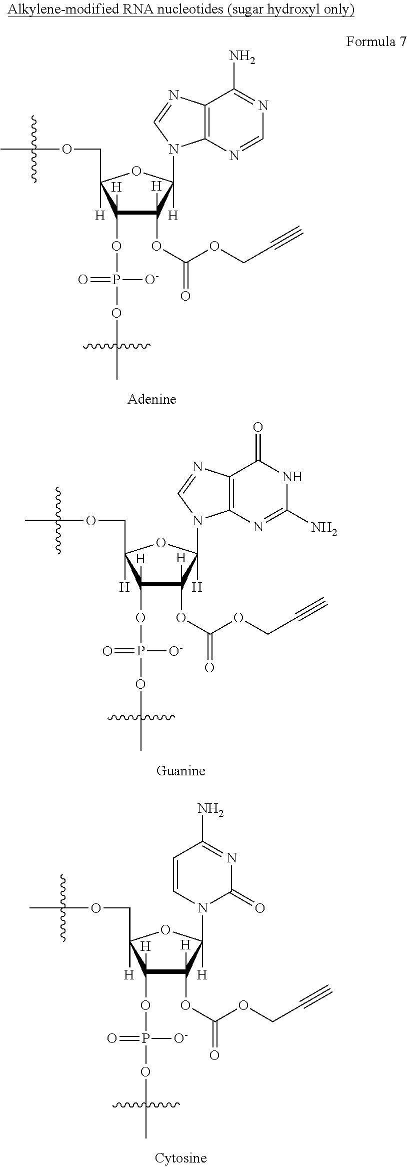

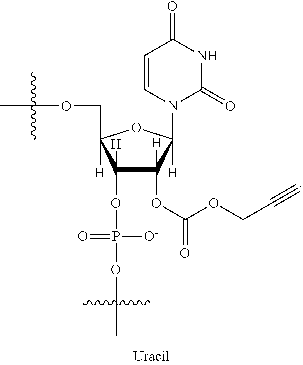

In embodiments where the biomolecule is an RNA, the alkyne-containing electrophilic reagent is propargyl chloroformate or the like, and only the sugar hydroxyl has been alkyne-modified, Formula 8 illustrates the product of the cycloaddition reaction between an azide-containing polymer (e.g., PEG azide terminated with a methoxy group or a targeting group) and the alkyne group appended to adenine, guanine, cytosine, and uracil.

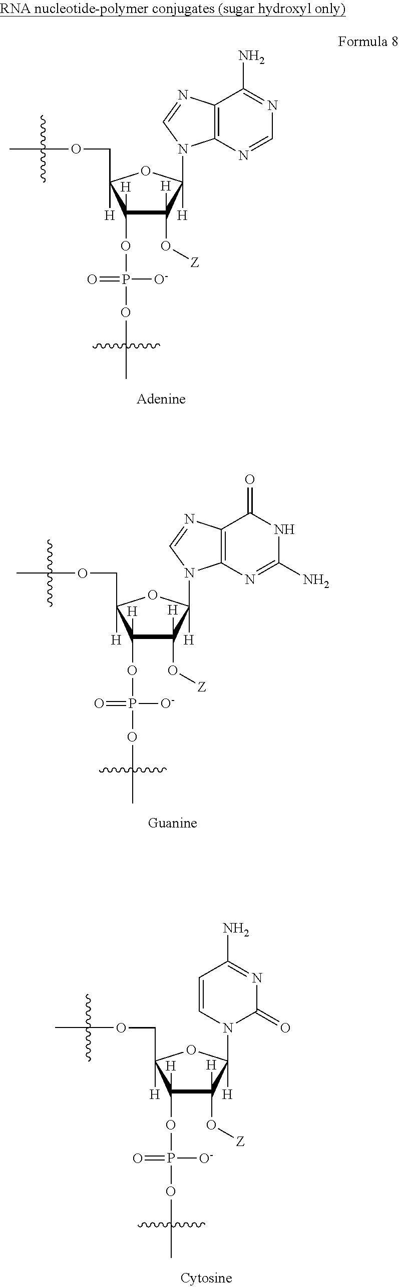

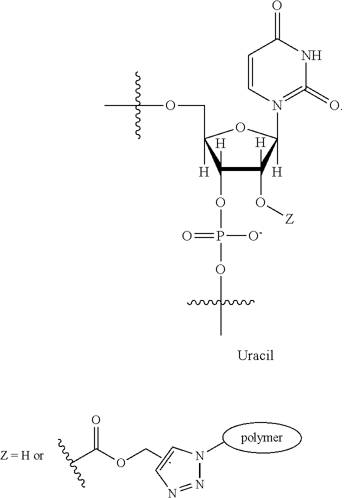

In other embodiments, the above-described method is analogously applied to DNAs. Natural DNA incorporated thymine, which, in embodiments where the biomolecule is a DNA and the alkyne-containing electrophilic reagent is propargyl chloroformate or the like, can be modified to form the product illustrated in Formula 9.

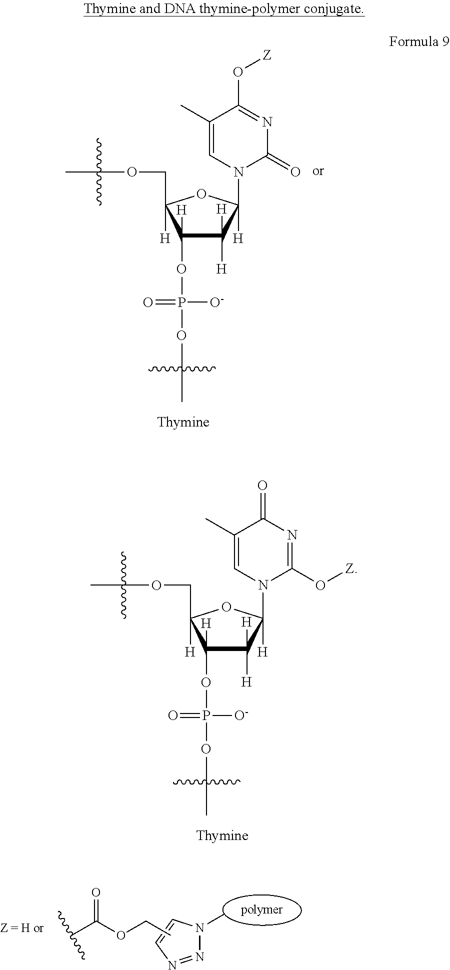

It will be understood that the methods may be analogously applied to biomolecules besides DNAs and RNAs, as described above.

### Kits

In another aspect of the disclosure, a kit suitable for preparing the biomolecule-polymer conjugate of the disclosure is provided, the kit comprising an alkyne-containing electrophilic reagent in a first container, an azide-containing biocompatible polymer in a second container, and instructions for their use.

In certain embodiments, the alkyne-containing electrophilic reagent is a carboxylic acid, an acid halide, a carboxylic acid anhydride, a carboxylic acid salt, a carboxylic acid ester, an isocyanate, a carbonate, a carbamate, or a chloroformate. In a particular embodiment, the alkyne-containing electrophilic reagent is

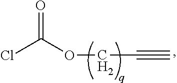

where q is an integer from 0 to about 20, from about 0 to about 10, from about 1 to about 10, from about 2 to about 10, from about 2 to about 8, from about 2 to about 5, or from about 2 to about 4. In various sub-embodiments, q is 0, 1, 2, 3, 4, 5, 6, 7, 8, 9, or 10. In a particular sub-embodiment, q is 2. In various sub-embodiments, each methylene group may be optionally substituted, or may itself be a different atom, such as NH, O, or S. In a particular embodiment, the alkyne-containing electrophilic reagent is a chloroformate, such as propargyl chloroformate

The azide-containing polymer can be any biocompatible polymer with an azide group. In certain embodiments, the azide-containing polymer imparts a stabilizing effect on the biomolecule. When n is greater than 1, the various polymers of Formula 1 can be the same or different. In various embodiments, the azide-containing polymer is independently anionically charged, cationically charged, or uncharged; hydrophobic, hydrophilic, or amphiphilic; or combinations thereof. In various embodiments, the azide-containing polymer is a homopolymer, a block copolymer, or a random copolymer. In certain embodiments, the azide-containing polymer is polydisperse or monodisperse. In various embodiments, the polydispersity index of the azide-containing polymer is from 1 to about 30, from 1 to about 10, from 1 to about 5, or from 1 to about 3. In certain embodiments the azide-containing polymer is linear. In certain embodiments the azide-containing polymer is branched.

In certain embodiments, the azide-containing polymer is a polyethylene glycol (PEG), a polyether, a poly(lactide), a poly(glycolide), a poly(lactide-co-glycolide), a poly(lactic acid), a poly(glycolic acid), a poly(lactic acid-co-glycolic acid), a polyanhydride, a polyorthoester, a polycarbonate, a polyetherester, a polycaprolactone, a polyesteramide, a polyester, a polyacrylate, a polymer of ethylene-vinyl acetate or another acyl substituted cellulose acetate, a polyurethane, a polyamide, a polystyrene, a silicone based polymer, a polyolefin, a polyvinyl chloride, a polyvinyl fluoride, a fluoropolymer, a polypropylene, a polyethylene, a cellulosic, a starch, a naturally occurring polymer, a poly(vinyl imidazole), a polyacetal, a polysulfone, a chlorosulphonate polyolefin, or a blend or copolymer thereof. In particular embodiments, the azide-containing polymer is PEG-azide.

In various embodiments, the azide-containing polymer has an average molecular weight of from about 200 to about 50,000, from about 200 to about 40,000, from about 200 to 30,000, from about 200 to about 20,000, from about 200 to about 10,000, from about 200 to about 5,000, from about 200 to about 4,000, from about 200 to about 3,000, from about 200 to about 2,000, from about 200 to about 1,000, or from about 200 to about 500. In various embodiments, the azide-containing polymer has an average molecular weight of from about 10,000 to about 50,000, from about 10,000 to about 40,000, from about 10,000 to about 30,000, or from about 10,000 to about 20,000. In a particular embodiment, the azide-containing polymer has an average molecular weight of from about 500 to about 5,000.

In certain embodiments, the azide-containing polymer is independently terminated with a non-functional group, such as a methyl or methoxy, or a functional group, such as a targeting group. In a particular embodiment, the targeting group is a folate. In certain embodiments, the azide-containing polymer is a mixture of non-functional terminated and functional terminated polymers. In certain embodiments, the mixture is a mixture of methoxy terminated and folate-terminated polymers, for example a mixture of methoxy-terminated PEG and folate-terminated PEG. In certain embodiments, the polymer is terminated with another biomolecule. In such an embodiment, the biomolecule-polymer conjugate is a networked biomolecule-polymer conjugate, each conjugate comprising more than one biomolecule.

In certain embodiments, the first container further comprises a first solvent. In certain embodiments, the first solvent is water, tetraethylene glycol dimethylether, dimethylsulfoxide, dimethylformamide, chloroform, dichloromethane, pyridine, acetone, ether, or a mixture thereof. In certain embodiments, the first solvent is a mixture of water and one or more of tetraethylene glycol dimethylether, dimethylsulfoxide, dimethylformamide, chloroform, dichloromethane, pyridine, acetone, or ether. In a particular embodiment, the first container does not comprise water. In a particular embodiment, the first solvent is water.

In certain embodiments, the second container further comprises a second solvent. In various embodiments, the second solvent is methanol, ethanol, propanol, isopropanol, tetraethylene glycol dimethylether, dimethylsulfoxide, dimethylformamide, acetone, ether, water, or a mixture thereof. In a particular embodiment, the second solvent is water and one or more of methanol, ethanol, propanol, isopropanol, tetraethylene glycol dimethylether, dimethylsulfoxide, dimethylformamide, acetone, or ether. In a particular embodiment, the second solvent is water.

The kit optionally further comprises one or more containers comprising the following: a base, a metal catalyst, a solvent, a purification column, a filter, a drying agent, a mixing vessel, a magnetic stirbar, and a filtration vessel.

In a particular embodiment, the kit comprises instructions to (1) dissolve the biomolecule in a first solvent (optionally provided with the kit); (2) add the alkyne-containing electrophilic reagent to the solution of the biomolecule; (3) optionally add a base to the solution of the biomolecule and alkyne-containing electrophilic reagent; (4) stir for between 30 minutes and 8 hours, or for between 1 hour and 2 hours; (5) remove solvent, alkyne-containing electrophilic reagent, and optional base; (6) dissolve the alkyne-modified biomolecule in a second solvent (optionally provided with the kit); (7) add the azide-containing polymer; (8) optionally add the metal catalyst; (8) stir for between 1 and 24 hours, or for about 2 hours, at room temperature or at a temperature from 35° C. to about 80° C.; (9) optionally concentrate the reaction mixture; (10) optionally purify using filter or column (optionally provided with the kit). The final product of the kit may be used in a cell based assay or as a diagnostic tool for laboratory use.

The disclosure will be further understood by the following non-limiting examples.

### Example 1

### Alkyne-Modification of an Oligonucleotide

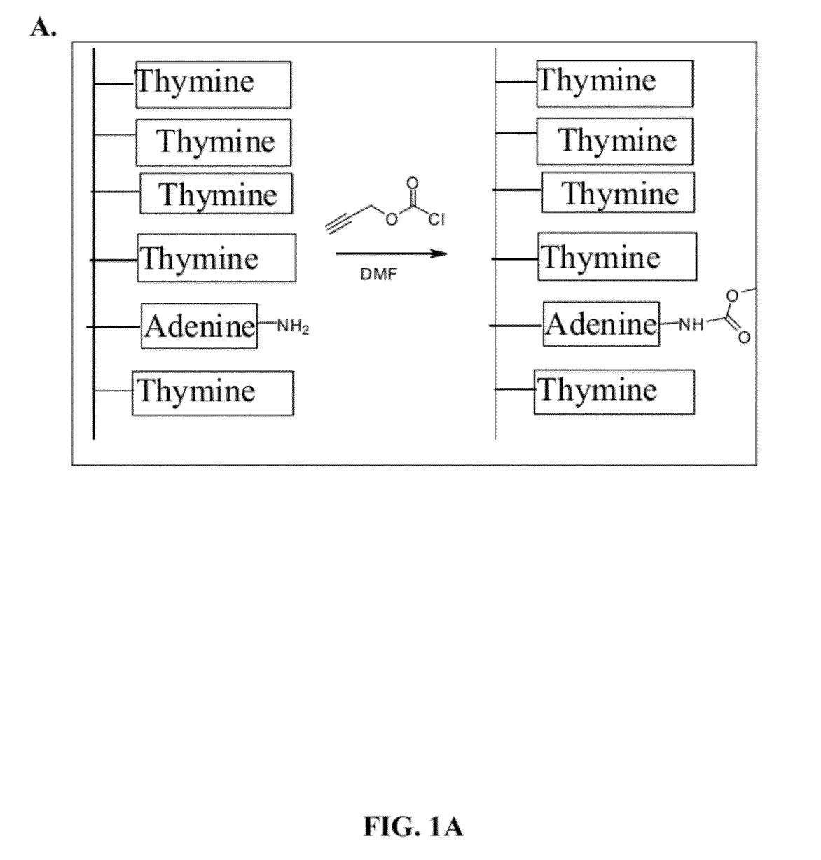

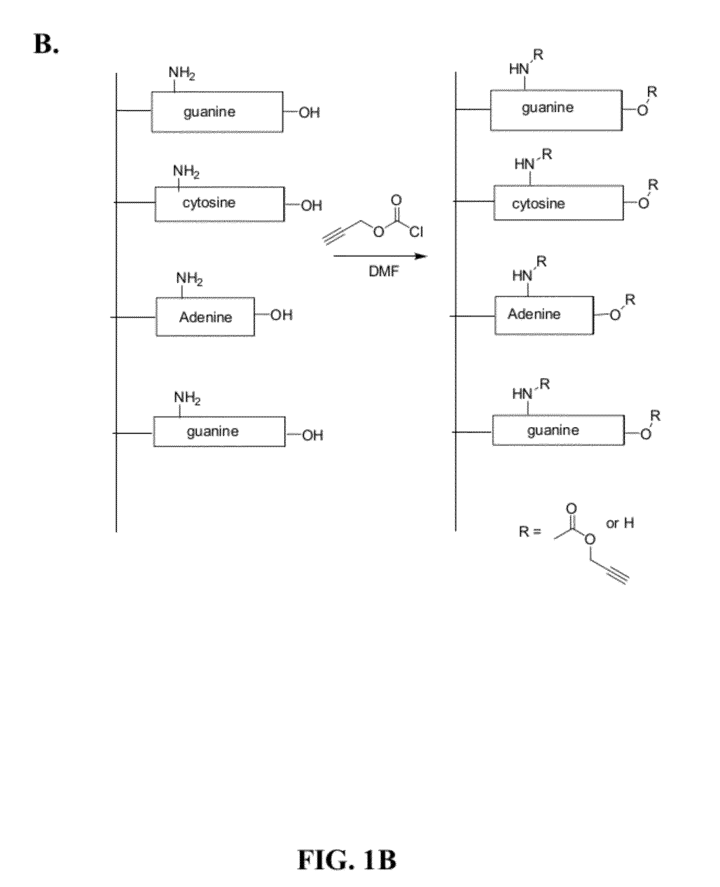

A model oligonucleotide, 5′-TTTTATTTTATTTTATTTTA-3′ (SEQ ID NO:1), can be modified according to the method of the disclosure. The model oligonucleotide (1 mg) is dissolved in 20 μL of dimethyl formamide (DMF) at room temperature. To this mixture is added propargyl chloroformate (1.8 μL), and the reaction mixture is allowed to stir for 2 hours. The reaction mixture is then concentrated under N₂ and the residue analyzed by Nuclear Magnetic Resonance (NMR) spectroscopy. While FIG. 1A only shows alkyne modification at one site, it is understood that such modification may occur at every modifiable site on the biological molecule. Therefore, in the model oligonucleotide, alkyne modification may occur at every adenine site.

FIG. 1B shows an oligonucleotide segment in which each nucleic acid base is alkyne-modified.

### Example 2

### Conjugation of Alkene-Modified Biomolecule to PEG Azides

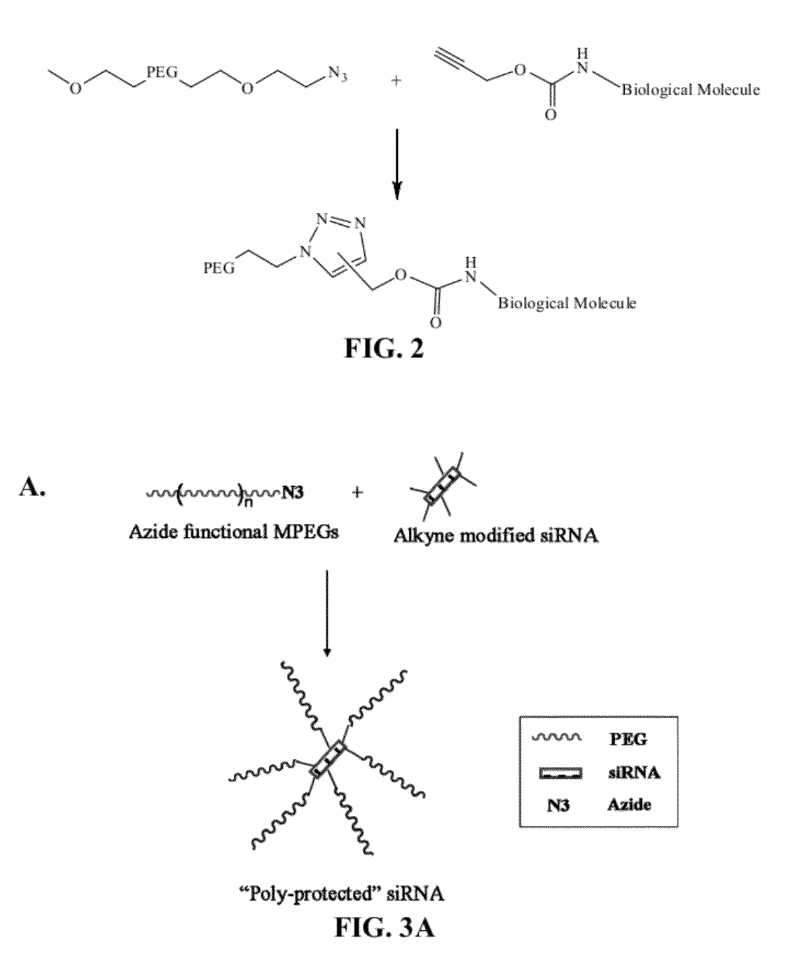

Click chemistry was first coined by Smalley et al. in 2001. The method of the disclosure makes use of the click chemistry Azide-Alkyne Huisgen Cycloaddition reaction. This “click reaction,” cleanly and efficiently attached an azide functional polymer to an alkyne-modified biomolecule. FIG. 2 illustrates an embodiment of the method using methoxy-terminated PEG-azide and a biomolecule modified with propargyl chloroformate or the like.

The method of the disclosure can be conducted as a “one-pot” reactions without isolation or cleaning of the products from the alkyne modification reaction. A representative reaction is as follows: the alkyne-modified biomolecule is dissolved in 30 μL of DMF. PEG azide is added to this mixture and the reaction mixture is allowed to stir for 2 hours at room temperature. The reaction mixture is concentrated under N₂ and analyzed by NMR spectroscopy.

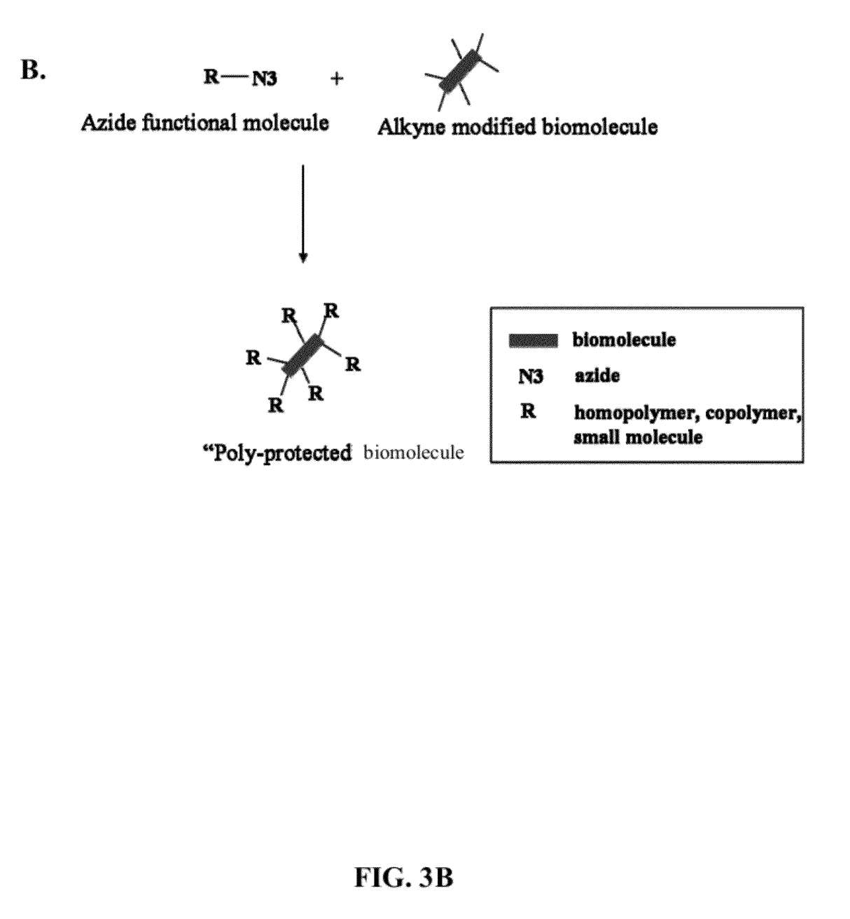

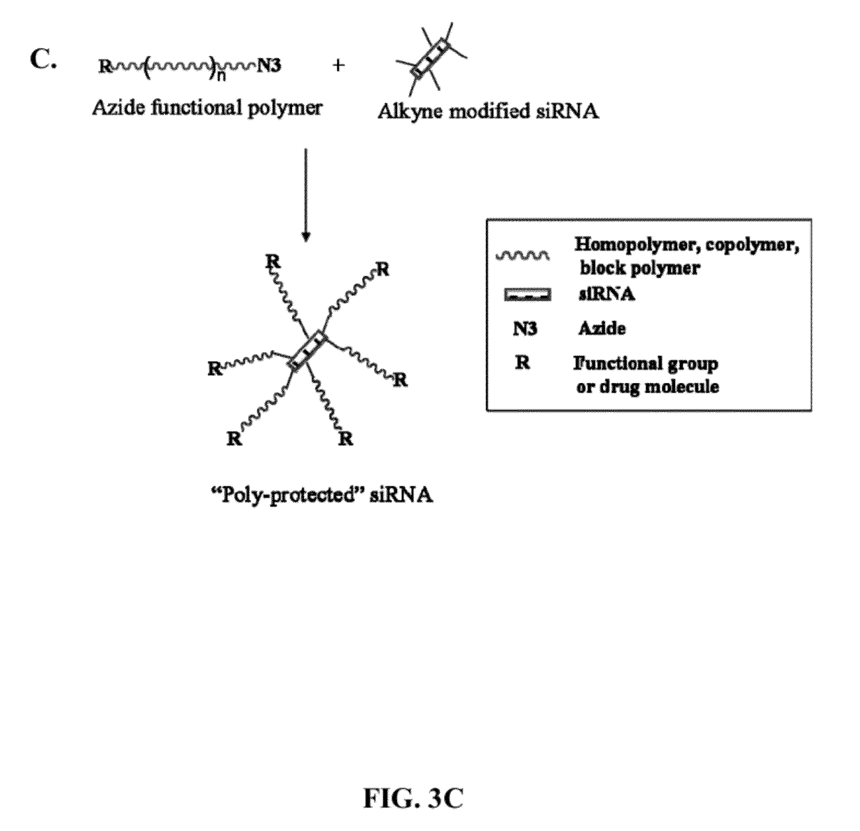

FIG. 3A illustrates an embodiment of the method. Using click chemistry, the biomolecule, in this case siRNA, is alkyne-modified at several locations along the nucleotide sequence. Several azide functional PEG chains are attached to the modified siRNA at the alkyne modification sites via the click reaction, creating an siRNA-polymer conjugate comprising multiple PEG strands.

### Example 3

### Conjugation of Alkyne-Modified Proteins to PEG Azides

Hemoglobin (1 mg) is dissolved in 30 μL of DMF at room temperature. To this mixture is added propargyl chloroformate (1.8 μL), and the reaction mixture is allowed to stir for 2 hours. The reaction mixture is then concentrated under N₂ and the residue analyzed by NMR spectroscopy. It is understood that such modification may occur at every modifiable site on the biological molecule, for example at a cysteine SH (e.g., Cys34). The alkyne-modified hemoglobin is then dissolved in 30 μL of DMF. PEG azide (10 μL) is added to this mixture and the reaction is allowed to stir for 2 hours at room temperature. The reaction mixture is concentrated under N₂ and analyzed by NMR spectroscopy.

FIG. 3B illustrates an embodiment of the method. Using click chemistry, the biomolecule, in this case hemoglobin, is modified at several locations. Azide-containing molecules (e.g., homopolymers, copolymers, or other small molecules) are attached to the modified biomolecule at the modification sites via the click reaction, creating a biomolecule conjugate comprising multiple conjugates homopolymers, copolymers, or other small molecules.

Hemoglobin is useful as a blood substitute, and thus the hemoglobin-polymer conjugates of the disclosure may be useful as prodrugs of hemoglobin. See, e.g., P. W. Buehler et al., Biomaterials, 2010, 31, 3723-3735.

### Example 4

### Conjugation of Alkene-Modified Peptides to PEG Azides

The peptide PYY (0.25 mg) is dissolved in 254 of DMF at room temperature. To this mixture is added propargyl chloroformate (1.8 μL), and the reaction mixture is allowed to stir for 2 hours. The reaction mixture is then concentrated under N₂ and the residue analyzed by NMR spectroscopy. It is understood that such modification may occur at every modifiable site on the biological molecule, for example at a tyrosine phenolic OH. The alkyne-modified peptide is then dissolved in 25 μL of DMF. PEG azide (1 μL) is added to this mixture and the reaction mixture is allowed to stir for 2 hours at room temperature. The reaction mixture is concentrated under N₂ and analyzed by NMR spectroscopy. FIG. 3B is also representative of this reaction.

PYY has been implicated in the treatment of obesity, and thus PYY-polymer conjugates of the disclosure may be useful prodrugs of PYY. See Marianne T. Neary et al., Pharmacology & Therapeutics, 2009, 124(1), 44-56.

### Example 5

### Conjugation of Alkyne-Modified Polysaccharide PEG Azides

The polysaccharide chitosan (1 mg) is dissolved in 204 of DMF at room temperature. To this mixture is added propargyl chloroformate (2 μL), and the reaction mixture is allowed to stir for 2 hours. The reaction mixture is then concentrated under N₂ and the residue analyzed by NMR spectroscopy. It is understood that such modification may occur at every modifiable site on the biological molecule, such as a glucosamine NH₂ or CH₂OH. The alkyne-modified chitosan from the modification reaction is dissolved in 30 μL of DMF. PEG azide (2 μL) is added to this mixture and the reaction mixture is allowed to stir for 2 hours at room temperature. The reaction mixture is concentrated under N₂ and analyzed by NMR spectroscopy. FIG. 3B is also representative of this reaction.

Chitosan may be useful for the treatment of various diseases, for example those characterized by over-expression of folic acid receptor cells, and thus chitosan-polymer conjugates of the disclosure may be useful as prodrugs of chitosan. See, e.g., U.S. Patent Application Publication No. 2009/324726 (“Fernandes et al.”).

### Example 6

### Conjugation of Alkyne-Modified Nucleotide to PEG Azides

The nucleotide adenosine triphosphate (ATP) (1 mg) is dissolved in 30 μL of DMF at room temperature. To this mixture is added propargyl chloroformate (1 μL), and the reaction is allowed to stir for 2 hours. The reaction mixture is then concentrated under N₂ and the residue analyzed by NMR spectroscopy. It is understood that such modification may occur at every modifiable site on the biological molecule, for example a sugar OH or the adenine NH₂. The alkyne-modified nucleotide from the modification reaction is dissolved in 30 μL of DMF. PEG azide (6 μL) is added to this mixture and the reaction is allowed to stir for 2 hours at room temperature. The reaction mixture is concentrated under N₂ and analyzed by NMR spectroscopy. FIG. 3B is also representative of this reaction.

### Example 7

### Alkyne-Modification of Oligonucleotide and Conjugation to Azide-Containing Polymer

An RNA or DNA oligonucleotide (0.1 mg) is dissolved in 20 μl of tetraethylene glycol dimethylether at room temperature. This is followed by the addition of 5 to 20 μl of diisopropylethylamine at room temperature. Propargyl chloroformate (0.5 μl) is then added, and the reaction mixture is allowed to stir for approximately 30 minutes. All volatile components are removed. Methanol (20 μl) is added. The azide-containing polymer (1 to 3 μl or 1 to 3 mg) is added and the reaction mixture stirred for approximately 1 hour. All volatiles are removed. The resulting product is then analyzed by HPLC.

### Example 8

### Preparation of Azide-Containing Polymer Terminated with a Targeting Group

The carboxylic acid of folate (300 mg) is first activated by reacting with N-hydroxysuccimide (NHS) and N,N′-Dicyclohexylcarbodiimide (DCC) in dimethyl sulfoxide (DMSO) stirred for 18 hours, filtered and washed with 30% acetone-ether to give the corresponding activated ester. This activated ester is then dissolved in dry pyridine and stirred with monoamine PEG azide for 18 hours. The pyridine is evaporated and the resulting mixture chromatographed to give the folate functionalized PEG azide. This folate functionalized PEG azide can be attached to a biomolecule as previously mentioned.

FIG. 3C illustrates an embodiment of the disclosure where the alkyne-modified biomolecule (e.g., siRNA) is conjugated to an azide-containing polymer terminated with a functional group such as a targeting group. The azide-containing polymers terminated with a functional group are conjugated to the alkyne-modified sites on the biomolecule via the click reaction, creating a biomolecule-polymer conjugate comprising multiple polymer strands terminated with a functional group.

### Example 9

### Conjugation of Alkyne-Modified Oligonucleotide to PEG Azides to Form Networks

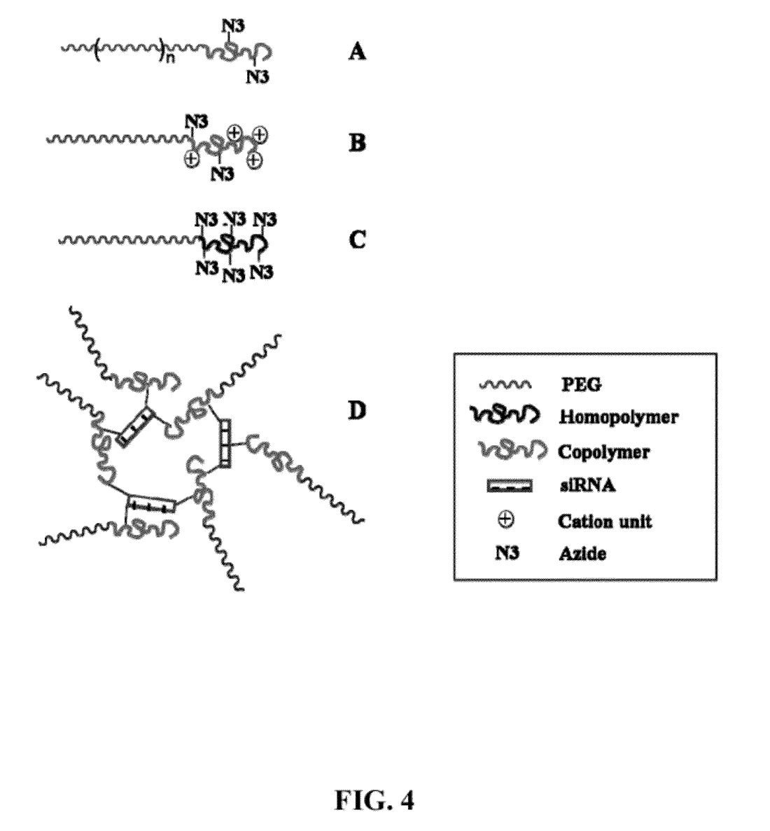

As illustrated FIG. 4, in one embodiment, polymer chains (e.g., PEG) of varying lengths are bonded to a copolymer having multiple azide functional sites (A). In another embodiment, the copolymer includes cationic groups along the backbone (B). In another embodiment, the polymer chains (e.g., PEG) of varying lengths can also be bonded to a multifunctional azide homopolymer segment (C). An alkyne-modified biomolecule is then conjugated to the polymer having multiple azide groups (A, B, or C), via the click reaction, to form a network of biomolecules and polymers (D). The additional azide functionalities on the polymers allows each polymer to form bonds with multiple biomolecules creating a network of bonded biomolecules and polymers.

It was originally suspected that such network formations would provide improved protection for the oligonucleotides. Work with the PCR primers showed that the networks show extended protection times of the functional oligonucleotide. Protection of biological molecules with multiple monofunctional azides and no additional networking is the preferred method and was studied most extensively in protection studies because this research has shown that it provides adequate protection and has the advantages of 1) simple polymer reactants; 2) simple separation of unreacted monomer (via dialysis); and 3) the reaction is less prone to leaving unreacted alkyne groups on the modified oligonucleotides. However, the networks have been shown to provide longer term protection of the oligonucleotides and can be used if they are found to be superior in specific cases (or may have potential as extended release systems).

### Example 10

### Study of Stability and Resistance to Degradation of an Oligonucleotide-Polymer Conjugate

Nucleases and proteases are common and result in extremely short half-life for biomolecules not protected from the in vivo environment. In addition, carboxylesterases (CES) are known to be present in many cancerous tumor cells. Therefore, the ability of the biomolecule-polymer conjugates of the disclosure to withstand degradation in the presence of fetal calf serum (FCS) and either DNase I, or 51 nuclease is investigated, as well as the ability of carboxylesterase 1 to degrade the biomolecule-polymer conjugates and release the biomolecule.

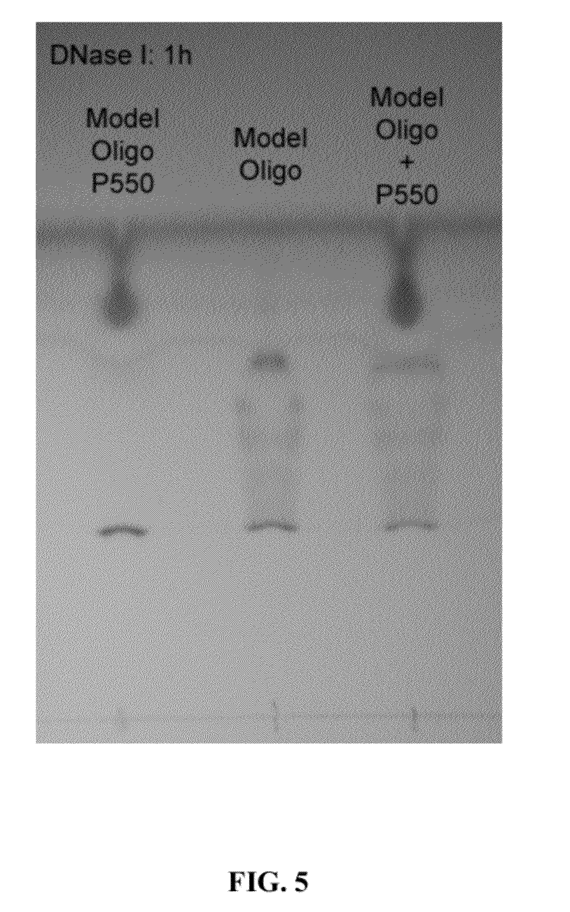

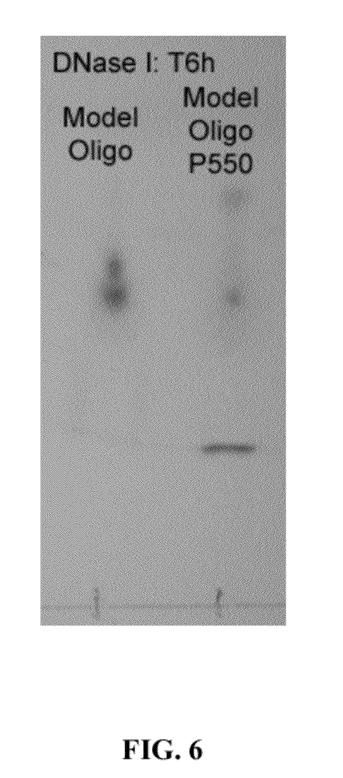

Concentrations of treatments were as follows: FCS (30%), DNase I (30 units), 51 nuclease (10 units). Digestions were conducted at 37° C. and monitored by either thin layer chromatography or gel electrophoresis. FIG. 5 shows TLC results under UV light showing DNase I digestion after one hour of the model oligonucleotide of Example 1 conjugated to MPEG550 (methoxy-terminated PEG, MW 550), the model oligonucleotide alone, and a blend of the model oligonucleotide and MPEG550. As shown in FIG. 5, it was found that the oligonucleotide-MPEG conjugate was resistant to DNase I treatment compared to the native model oligonucleotide and the blend of native model oligonucleotide and MPEG550.

FIG. 6 shows TLC results under UV light showing DNase I digestion after six hours of the model oligonucleotide and the oligonucleotide-MPEG conjugate. As can be seen in FIG. 6, the native model oligonucleotide was completely digested after 6 hours while the oligonucleotide-MPEG conjugate remained intact.

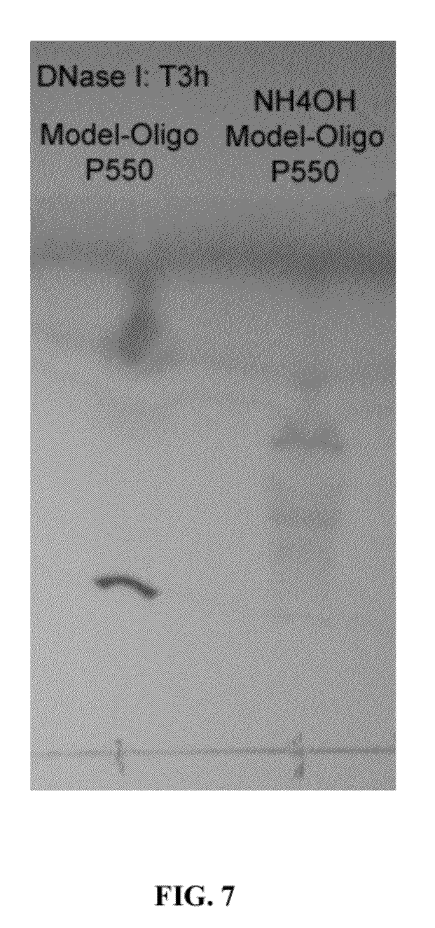

In addition, the chemical degradation of the oligonucleotide-MPEG conjugate using ammonium hydroxide (NH₄OH) (to cleave the L-X bond) allowed for degradation by DNase I after 3 hours, as shown in FIG. 7. This demonstrates that the conjugation of the biomolecule protects the biomolecule from digestion, but that the conjugation is reversible.

### Example 11

### Functional Sequence Results K-ras

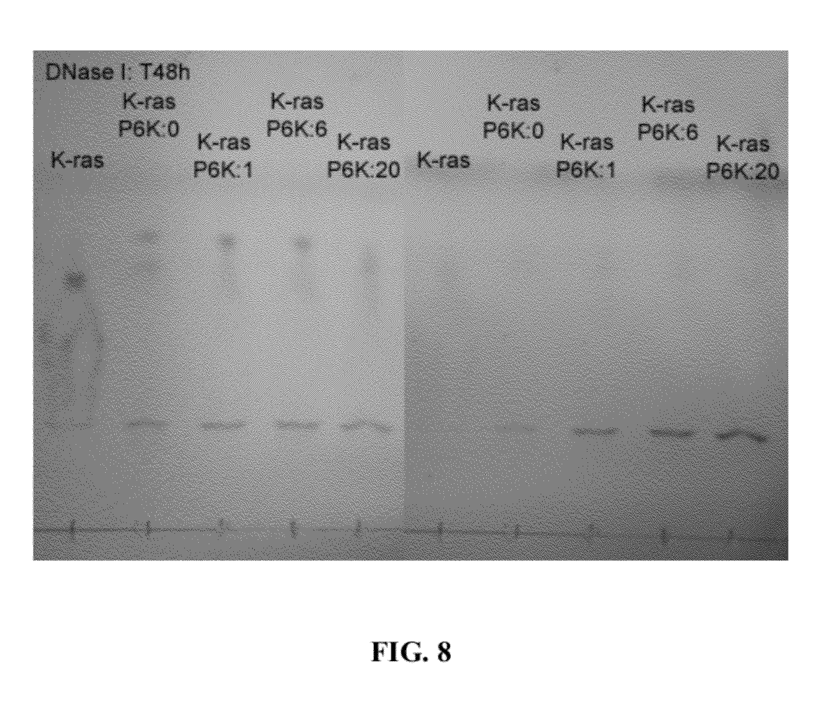

The functional K-ras was modified according to the method of Example 7 using MPEG6k (methoxy-terminate PEG, MW 6000) at low (one equivalent MPEG6k, i.e., n is about 1), medium (six equivalents MPEG6k, i.e., n is about 6), and high substitution (excess MPEG6k, i.e., n is about 11-30), and evaluated for stability in the presence of DNase I. FIG. 8 shows TLC results under UV light (left) and vanillin stained (right) showing DNase I digestion after 48 hours of K-ras, a blend of K-ras and MPEG6k, Kras conjugated to one equivalent MPEG6k, i.e., n is about 1, K-ras conjugated to about six equivalents MPEG6k, i.e., n is about 6, and K-ras conjugated to excess MPEG6k, i.e., n is about 11-30. As shown in FIG. 8, even a small amount of substitution aids in the prevention of degradation in DNase I over the control. In addition, a high amount of substitution gives significant protection against degradation. This may allow for a selective level of modification in order to tailor the circulation time desired for a given therapy.

### Example 12

### Functional Sequence Results PCR Primer

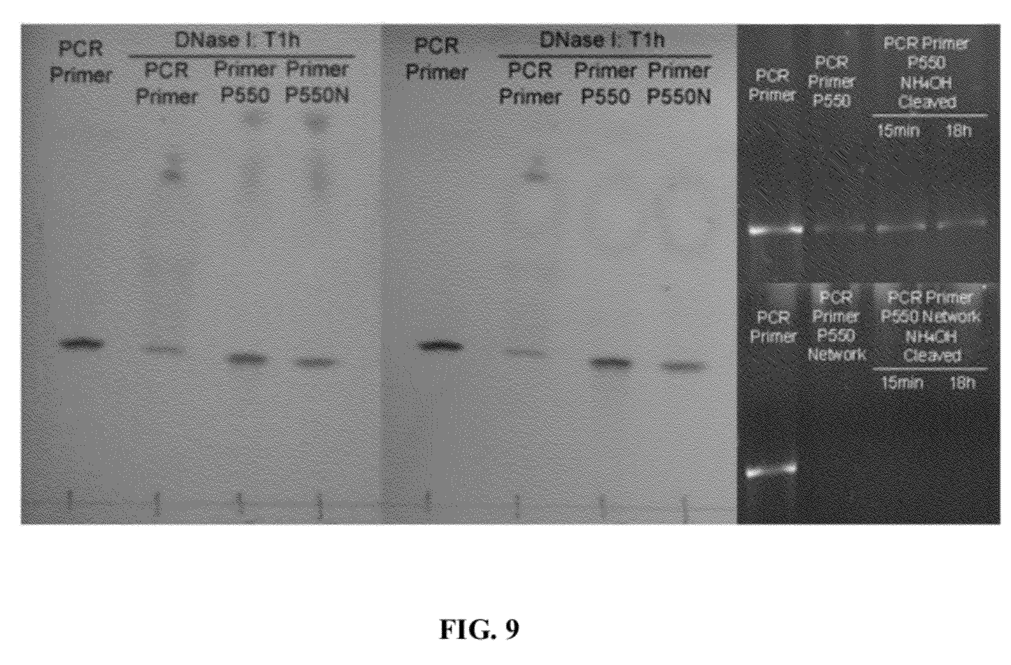

The retention and recovery of the original functionality of the biomolecule is a characteristic of the conjugates of the disclosure. In order to test the ability of an oligonucleotide-polymer conjugate of the disclosure to functionally bind its complementary sequence, PCR primers were utilized. The universal bacterial primers 8F (5′-AGAGTTTGATCCTGGCTCAG-3′, SEQ ID NO:2) and 1392R (5′-ACGGGCGGTGTGTACA-3′, SEQ ID NO:3) were used to amplify a portion of the bacterial 16S ribosomal RNA gene. The 8F primer conjugated to MPEG-550 networked with MPEG-550 were prepared according to Examples 7 and 9 above, and subjected to DNase I degradation studies. FIG. 9 shows TLC results under UV light (left) and vanillin stained (middle) showing DNase I digestion after one hour of PCR primer (control), PCR primer (digest), the MPEG550 conjugate, and the MPEG550-networked-conjugate.

As shown in FIG. 9, the MPEG550 conjugate and the MPEG550-networked-conjugate were resistant to nuclease degradation. The MPEG550 conjugate and the MPEG550-networked-conjugate were then treated with NH₄OH to release the PCR primers from the MPEG550 via chemical degradation and PCR amplification was then performed. Each 50-μl PCR mixture contained 1 μl template DNA (either E. coli. or an environmental isolate belonging to the genus Streptomonospora), 2 U Taq DNA polymerase (Eppendorf), 1×Taq buffer, 2.75 μM Mg(OAc)₂, 1×Taq Master PCR Enhancer, each deoxynucleoside triphosphate at a concentration of 20 μM, and each primer at a concentration of 0.4 μM. The PCR conditions were 85° C. for 5 min, 30 cycles of 94° C. for 45 s, 55° C. for 1 min, and 72° C. for 90 s, with a final 7 min extension at 72° C. The right side of FIG. 8 shows gel electrophoresis results in a 1% agarose gel showing the PCR amplification products of PCR primer (unmodified 8F primer), MPEG550 conjugate, and the MPEG550 conjugate cleaved in NH₄OH for 15 minutes and 18 hours. It was found that the MPEG550 conjugates were functional and yielded the appropriate size amplification product, as did the NH₄OH treated MPEG550 conjugate, however no amplification product was detected for the MPEG550-networked-conjugate. Nevertheless, FIG. 9 also shows that the MPEG550 conjugate and MPEG550-networked-conjugate show excellent protection when exposed to DNase I over the unmodified PCR primer.

### Example 13

### Functional Sequence Results Salmon Sperm DNA

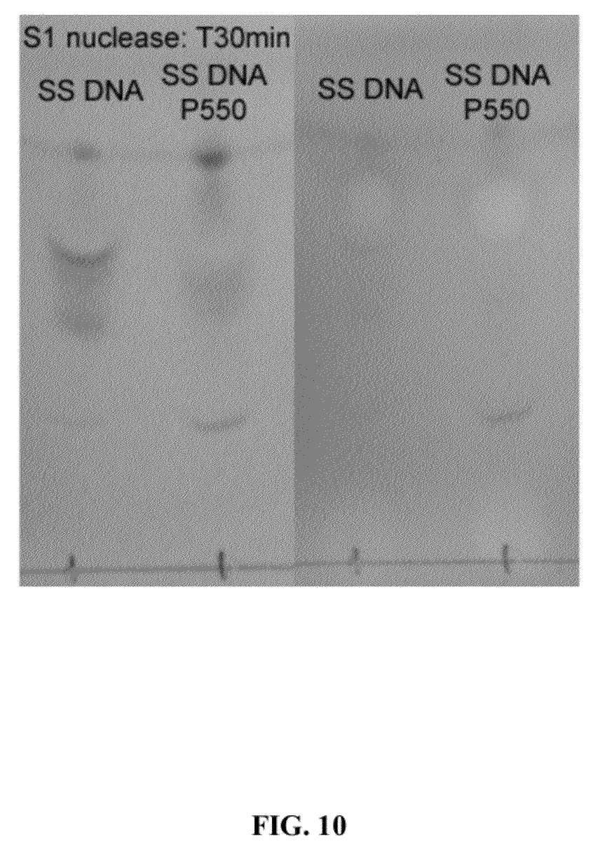

Salmon sperm (SS) DNA was used as an example biomolecule. An SS-MPEG550 conjugate was prepared according to Example 7 and digested with 51 Nuclease. FIG. 10 shows TLC results under UV light (left) and vanillin stained (right) showing 51 Nuclease digestion after 30 minutes of SS DNA (control) and SS-MPEG550 conjugate. As can be seen in FIG. 10, when exposed to the very aggressive 51 Nuclease, the SS-MPEG550 conjugate is stable while the native SS DNA is almost completely digested.

### Example 14

### Functional Sequence Results siRNA

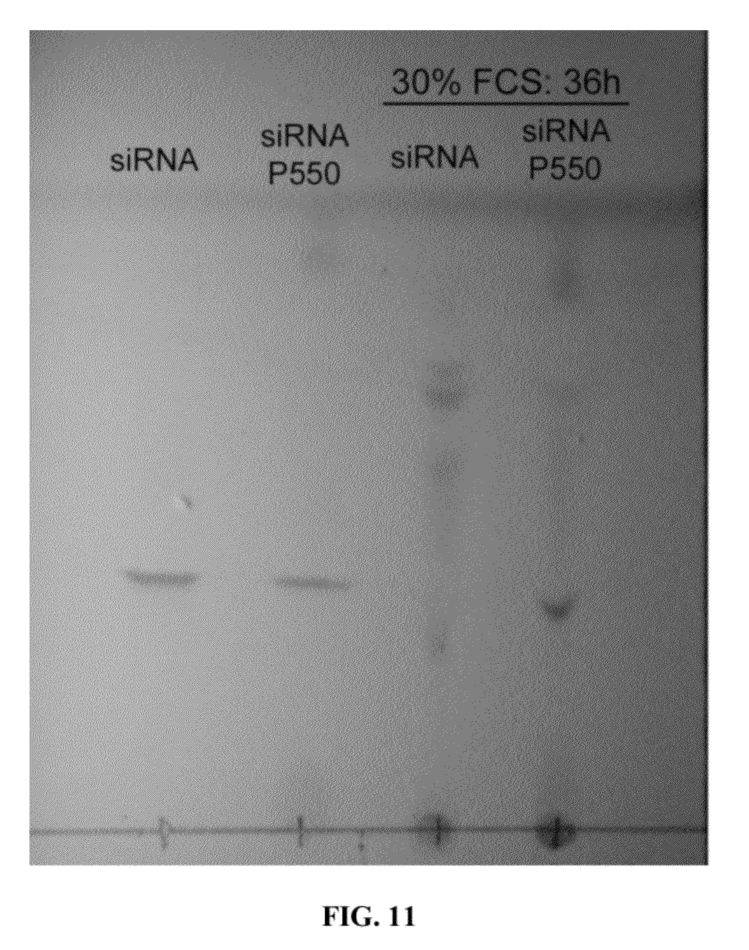

Functional p53 siRNA was conjugated to MPEG 550 according to the method of Example 7 and evaluated for stability against FCS. FIG. 11 shows TLC results under UV light showing FCS digestion after 36 hours with samples including siRNA (control), siRNA-MPEG550 conjugate (control), siRNA (digest), and siRNA-MPEG550 conjugate (digest). As can be seen in FIG. 11, when the siRNA-MPEG550 conjugate is exposed to 30% FCS for 36 hours, conditions in which the siRNA alone is completely degraded, the siRNA-MPEG550 conjugate remains stable.

The examples set forth above are provided to give those of ordinary skill in the art with a complete disclosure and description of how to make and use the claimed embodiments, and are not intended to limit the scope of what is disclosed herein. Modifications that are obvious to persons of skill in the art are intended to be within the scope of the following claims. All publications, patents, and patent applications cited in this specification are incorporated herein by reference as if each such publication, patent or patent application were specifically and individually indicated to be incorporated herein by reference.

1. A biomolecule-polymer conjugate of Formula 1: 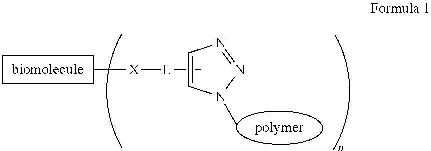
wherein
X is independently O, NH, NR, or S, and X and R are independently atoms of the biomolecule;
L is independently a 1-20 atom linear or branched linker;
n is an integer;
the polymer is a biocompatible polymer; and
wherein the X-L bond is degradable.

2. (canceled)

3. The conjugate of claim 1 or 2, wherein the biomolecule is a peptide, polypeptide, protein, polysaccharide, nucleic acid, nucleotide, amino acid, polynucleotide, or a mixed group thereof.

4. The conjugate of claim 1, wherein the biomolecule is a DNA or RNA molecule that comprises about 2 to about 30 bases.

5. The conjugate of claim 1 wherein the biomolecule is an antisense molecule, siRNA, or miRNA.

6. (canceled)

7. (canceled)

8. The conjugate of claim 4, wherein X-L is 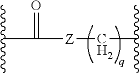
wherein Z is O or NH, and q is an integer from 0 to 20.

9. The conjugate of claim 8, wherein the biocompatible polymer is a polyethylene glycol (PEG), a polyether, a poly(lactide), a poly(glycolide), a poly(lactide-co-glycolide), a poly(lactic acid), a poly(glycolic acid), a poly(lactic acid-co-glycolic acid), a polyanhydride, a polyorthoester, a polycarbonate, a polyetherester, a polycaprolactone, a polyesteramide, a polyester, a polyacrylate, a polymer of ethylene-vinyl acetate or another acyl substituted cellulose acetate, a polyurethane, a polyamide, a polystyrene, a silicone based polymer, a polyolefin, a polyvinyl chloride, a polyvinyl fluoride, a fluoropolymer, a polypropylene, a polyethylene, a cellulosic, a starch, a naturally occurring polymer, a poly(vinyl imidazole), a polyacetal, a polysulfone, a chlorosulphonate polyolefin, or a blend or copolymer thereof.

10. The conjugate of claim 9, wherein the biocompatible polymer is methoxy-terminated polyethylene glycol or folate-terminated polyethylene glycol.

11. (canceled)

12. The conjugate of claim 4, wherein n is from about 1 to about 30.

13. (canceled)

14. The conjugate of claim 1, wherein the conjugate is substantially free of copper.

15. A method of preparing a biomolecule-polymer conjugate of Formula 1: 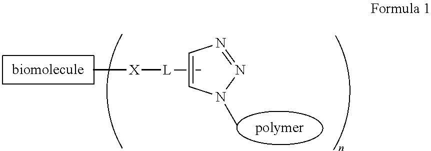
wherein
X is independently O, NH, NR, or S, and X and R are independently atoms of the biomolecule;
L is independently a 1-20 atom linear or branched linker;
n is an integer;
the polymer is a biocompatible polymer; and
wherein the X-L bond is degradable;
the method comprising
a. reacting the biomolecule with an alkyne-containing electrophilic reagent to form a modified biomolecule of Formula B: 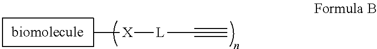
wherein the biomolecule, X, L, and n are as defined above; and
b. reacting the modified biomolecule of Formula B with a polymer or mixture of polymers of Formula C: 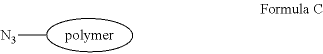
wherein the polymer is as defined above.

16. The method of claim 15, wherein the biomolecule is a peptide, polypeptide, protein, polysaccharide, nucleic acid, nucleotide, amino acid, polynucleotide, or a mixed group thereof.

17. The method of claim 15, wherein the alkyne-containing electrophilic reagent is: 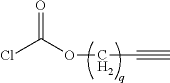
wherein q is an integer from 0 to 20.

18. (canceled)

19. The method of claim 17, wherein the biocompatible polymer is a polyethylene glycol (PEG), a polyether, a poly(lactide), a poly(glycolide), a poly(lactide-co-glycolide), a poly(lactic acid), a poly(glycolic acid), a poly(lactic acid-co-glycolic acid), a polyanhydride, a polyorthoester, a polycarbonate, a polyetherester, a polycaprolactone, a polyesteramide, a polyester, a polyacrylate, a polymer of ethylene-vinyl acetate or another acyl substituted cellulose acetate, a polyurethane, a polyamide, a polystyrene, a silicone based polymer, a polyolefin, a polyvinyl chloride, a polyvinyl fluoride, a fluoropolymer, a polypropylene, a polyethylene, a cellulosic, a starch, a naturally occurring polymer, a poly(vinyl imidazole), a polyacetal, a polysulfone, a chlorosulphonate polyolefin, or a blend or copolymer thereof.

20. The method of claim 19, wherein the polymer or mixture of polymers of Formula C is methoxy-terminated PEG-azide.

21. (canceled)

22. The method of claim 19, wherein the polymer or mixture of polymers of Formula C is a mixture of methoxy-terminated PEG-azide and folate-terminated PEG-azide.

23. A kit for preparing the biomolecule-polymer conjugate of claim 1, comprising an alkyne-containing electrophilic reagent in a first container, a polymer or mixture of polymers of Formula C: 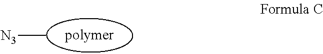
in a second container, and instructions for use.

24. The kit according to claim 23, wherein the alkyne-containing electrophilic reagent is: 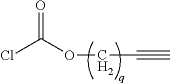
wherein q is an integer from 0 to 20.

25. The kit according claim 23, wherein the polymer or mixture of polymers of Formula C is methoxy-terminated PEG-azide.

26. The kit according to claim 23, wherein the polymer or mixture of polymers of Formula C is a mixture of methoxy-terminated PEG-azide and folate-terminated PEG-azide.

27. A biomolecule-polymer conjugate of Formula 1: 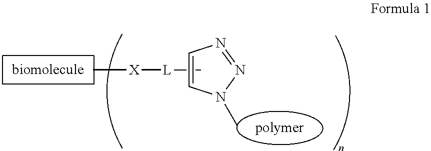
wherein
X is independently O, NH, NR, or S, and X and R are independently atoms of the biomolecule, and the biomolecule is an siRNA or miRNA;
L is independently a 1-20 atom linear or branched linker;
n is from about 11 to about 13;
the polymer is a mixture of methoxy-terminated PEG and folate-terminated PEG; and
wherein X-L is 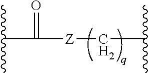
wherein Z is O or NH, and q is an integer from 0 to 20.


SEQUENCE LISTING (3 sequences)

SEQ ID NO: 1 - DNA, 20 bp, Artificial Sequence
  A model oligonucleotide
ttttattttattttatttta

SEQ ID NO: 2 - DNA, 20 bp, Artificial Sequence
  Universal bacterial primer 8F
agagtttgatcctggctcag

SEQ ID NO: 3 - DNA, 16 bp, Artificial Sequence
  Universal bacterial primer 1392R
acgggcggtgtgtaca